(19) United States
(12) Patent Application Publication
(10) Pub. No.: US 2010/0016400 A1
(43) Pub. Date: Jan. 21, 2010

(54) AZABICYCLIC MUSCARINIC RECEPTOR ANTAGONISTS

(76) Inventors: Naresh Kumar, Gurgaon (IN); Mohammad Salman, Princeton, NJ (US); Kirandeep Kaur, Gurgaon (IN); Anita Mehta, Plainfield (IL); Sudershan K. Arora, Pune (IN); Anita Chugh, New Delhi (IN)

Correspondence Address: Ranbaxy Inc., Intellectual Property Department, 600 College Road East, PRINCETON, NJ, 08540, US

(21) Appl. No.: 11/719,687

(22) PCT Filed: Nov. 18, 2005
(86) PCT No.: PCT/IB05/03459

§ 371 Date: Feb. 10, 2009

(30) Foreign Application Priority Data
Nov. 22, 2004 (IN) 2331/DEL/2004

(51) Int. Cl. A61K 31/403; C07D 209/52; C07D 409/06; A61P 11/00

(52) U.S. Cl. 514/412; 548/515; 548/465

(57) ABSTRACT

The present invention generally relates to muscarinic receptor antagonists, which are useful for treating various diseases of the respiratory, urinary and gastrointestinal systems mediated through muscarinic receptors. The invention also relates to processes for preparing compounds described herein, pharmaceutical compositions containing the disclosed compounds, and the methods for treating diseases mediated through muscarinic receptors.

### FIELD OF THE INVENTION

The present invention generally relates to muscarinic receptor antagonists, which are useful for treating various diseases of the respiratory, urinary and gastrointestinal systems mediated through muscarinic receptors. The invention also relates to processes for preparing compounds described herein, pharmaceutical compositions containing the disclosed compounds, and methods for treating diseases mediated through muscarinic receptors.

### BACKGROUND OF THE INVENTION

Muscarinic receptors, members of the G Protein Coupled Receptors (GPCRs), are composed of a family of 5 receptor sub-types (M₁, M₂, M₃, M₄ and M₅) and are activated by the neurotransmitter acetylcholine. These receptors are widely distributed on various organs and tissues and are critical to the maintenance of central and peripheral cholinergic neurotransmission. The regional distribution of these receptor sub-types in the brain and other organs has been documented (for example, the M₁ subtype is located primarily in neuronal tissues, such as cerebral cortex and autonomic ganglia; the M₂ subtype is present mainly in the heart where it mediates cholinergically induced bradycardia; and the M₃ subtype is located predominantly on smooth muscle and salivary glands (Nature, 323, p. 411 (1986); Science, 231, p. 527 (1987)).

A review in Current Opinions in Chemical Biology, 3, p. 426 (1999), as well as in Trends in Pharmacological Sciences, 22, p. 409 (2001) by Eglen et al., describes the biological potentials of modulating muscarinic receptor subtypes by ligands in different disease conditions, such as Alzheimer's Disease, pain, urinary disease condition, chronic obstructive pulmonary disease, and the like.

A review in J. Med. Chem., 43, p. 4333 (2000), by Felder et al, describes therapeutic opportunities for muscarinic receptors in the central nervous system and elaborates on muscarinic receptor structure and function, pharmacology and their therapeutic uses.

The pharmacological and medical aspects of the muscarinic class of acetylcholine agonists and antagonists are presented in a review in Molecules, 6, p. 142 (2001).

Birdsall et al, in Trends in Pharmacological Sciences, 22, p. 215 (2001) have also summarized the recent developments on the role of different muscarinic receptor subtypes using different muscarinic receptor of knock out mice. Annual Review of Pharmacological Toxicol., 41, p. 691 (2001), describes the pharmacology of the lower urinary tract infections. Almost all smooth muscle tissues express both M₂ and M₃ receptors, both of which have the functional role. M₂ receptors outnumber M₃ receptors by a proportion of approximately 4 to 1. Generally, M₃ receptors mediate the direct contractile effects of acetylcholine in the vast majority of smooth muscle tissues. M2 receptors, on the other hand, cause smooth muscle contraction indirectly by inhibiting sympathetically (β-adrenoreceptor)-mediated relaxation. Compounds that act as antagonists of muscarinic receptors have been used to treat several disease states associated with improper smooth muscle function, as well as in the treatment of cognitive and neurodegenerative disorders such as Alzheimer's disease. Until recently, most of these compounds have been non-selective for the various muscarinic receptor subtypes, leading to unpleasant anti-cholinergic side effects such as dry mouth, constipation, blurred vision, or tachycardia. The most common of these side-effects is dry mouth resulting from muscarinic receptor blockade in the salivary gland. Recently developed M₂ or M₃ specific antagonists have been shown to have reduced side effects. And also the side effects associated with oxybutynin, the nonselective antimuscarinic agent, are believed to be due to its affinity for the M₅ muscarinic receptor.

Evidence suggests that mechanistically, concurrent blockade of M₂ and M₃ receptors by sparing M₅ receptors could be therapeutically effective in the treatment of the disease states associated with smooth muscle disorders. Few M₂/M₃ selective and M₅ sparing antagonists have been developed.

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 93/16018 and WO96/33973 are other references of interest; WO 97/45414 is related to 1,4-disubstituted piperidine derivatives; WO 98/05641 describes fluorinated, 1,4-disubstituted piperidine derivatives; U.S. Pat. No. 5,397,800 discloses 1-azabicyclo[2.2.1]heptanes; U.S. Pat. No. 5,001,160 describes 1-aryl-1-hydroxy-1-substituted-3-(4-substituted-1-piperazinyl)-2-propanones; WO 99/43657 describes 2-arylethyl-(piperidin-4-ylmethyl)amine derivatives as muscarinic receptors antagonists; WO 01/090082 describes substituted 1-amino-alkyl lactams and their use as muscarinic receptor antagonists; WO 01/47893 describes azabicyclooctane derivatives useful in the treatment of cardiac arrhythmias; WO 01/42213 describes 2-biphenyl-4-piperidinyl ureas; WO 01/42212 describes carbamate derivatives. WO 01/90081 describes amino alkyl lactam; WO 02/53564 describes novel quinuclidine derivatives; WO 02/00652 describes carbamates derived from arylalkyl amines; WO 02/06241 describes 1,2,3,5-tetrahydrobenzo(c)azepin-4-one derivatives; U.S. Application No. 20030105071 describes thiazole and other heterocyclic ligands for mammalian dopamine, muscarinic and serotonin receptors and transporters, and method of use thereof; WO 03/033495 describes quinuclidine derivatives and their use as M₂ and/or M₃ muscarinic receptor antagonists; US2003/0171362 describes amino-tetralin derivatives as muscarinic receptor antagonists; US2003/0162780 describes 4-piperidinyl alkyl amine derivatives as muscarinic receptor antagonists; U.S. Pat. No. 5,179,108 disclose derivatives of 4-(aminomethyl)piperidine and their therapeutic applications; WO 03/048125 discloses aminotetralin derivatives as muscarinic receptor antagonists; WO 03/048124 discloses 4-piperidinyl alkylamine derivatives as muscarinic receptor antagonists; WO 2004/052857, WO 2004/067510 and WO 04/004629 disclose 3,6-disubstituted azabicyclo [3.1.0] hexane derivatives useful as muscarinic receptor antagonists; WO 04/005252 discloses azabicyclo derivatives as muscarinic receptor antagonists; WO 04/014853 and WO 04/014363 disclose derivatives of 3,6-disubstituted azabicyclohexane useful as muscarinic receptor antagonists; WO 2004/056810 discloses xanthine derivatives as muscarinic receptor antagonists; WO 2004/056811 discloses flazavate derivatives as muscarinic receptor antagonists; WO 2004/056767 discloses 1-substituted-3-pyrrolidine derivatives as muscarinic receptor antagonists; WO 2004/018422 disclose fluoro and sulphonylamino containing 3,6-disubstituted azabicyclo[3.1.0] hexane derivatives as muscarinic receptor antagonists; WO 2004/089900 disclose azabicyclo derivatives as muscarinic receptor antagonists. WO 2004/089898, WO 2004/089363, WO 2004/069835 and WO 2004/089899 disclose substituted azabicyclohexane derivatives as muscarinic receptor antagonists.

J. Med. Chem., 44, p. 984 (2002), describes cyclohexylmethylpiperidinyltriphenylpropioamide derivatives as selective M₃ antagonist discriminating against the other receptor subtypes.

J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds.

J. Med. Chem., 34. p. 3065 (1991), describes analogues of oxybutynin, synthesis and antimuscarinic activity of some substituted 7-amino-1-hydroxy-5-heptyn-2-ones and related compounds.

In view of the above, there remains a need for M₂ and/or M₃ and M₅ sparing muscarinic receptor antagonists useful in the treatment of disease states associated with improper smooth muscle function and respiratory disorders.

### SUMMARY OF THE INVENTION

In one aspect, provided are muscarinic receptor antagonists. Such muscarinic receptor antagonists can be useful as safe and effective therapeutic or prophylactic agents for treating various diseases of the respiratory, urinary and gastrointestinal systems. Also provided are processes for synthesizing such compounds.

In another aspect, provided are pharmaceutical compositions containing compounds described herein together with one or more acceptable carriers, excipients or diluents. Such pharmaceutical compositions can be useful for the treatment of various diseases of the respiratory, urinary or gastrointestinal systems.

Also provided are enantiomers, diastereomers, N-oxides, polymorphs, pharmaceutically acceptable salts and pharmaceutically acceptable solvates of compounds described herein, as well as metabolites having the same type of activity. Also provided are pharmaceutical compositions comprising metabolites, enantiomers, diastereomers, N-oxides, polymorphs, solvates or pharmaceutically acceptable salts of the compounds described herein, in combination with one or more pharmaceutically acceptable carrier and optionally included excipients.

Thus in one aspect, provided are compounds having the structure of Formula I,

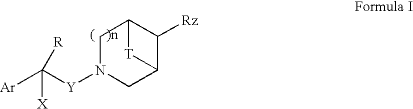

wherein
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl, cycloalkyl or aryl;
Y is —C(═O), —C(═S), —C(═Nacyl), —C(═N(NO₂)), —C(═CH(NO₂)), —C(═C(R₁)₂) or —CH₂—;
T is —(CH₂)ₘ, —CH(Q)CH₂, —CH(Q), —CH₂—O—CH₂;
Rz is hydrogen, hydroxy, alkoxy, hydroxyalkyl, aryloxy, —CHO, —CN, alkyl, alkenyl, alkynyl, cycloalkyl, carboxy, halogen, aryl, aralkyl, acyl, heteroaryl, heterocyclyl, heteroarylalkyl, heterocyclylalkyl, —(CH₂)ₖNRₓR_{y}, —SO₂R₂, —COOR₃, —C(═O)NRₓR_{y}, —NRₓR_{y}, —OC(═O)NRₓR_{y}, —NR₁C(═O)Rₓ or —NHC(═O)Rₓ; and
   n is an integer from 0-2, wherein when n is zero then n represents a direct bond);
      wherein
   Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time);
   R₁ is hydrogen, alkyl, alkenyl, alkynyl, cycloalkyl, aryl, heterocyclyl, heteroaryl, aralkyl, heteroarylalkyl or heterocyclylalkyl;
   m is an integer from 0-3, wherein T represents a direct bond when m is zero;
   Q is alkyl, alkenyl, alkynyl, cycloalkyl, aryl, heteroaryl, heterocyclyl, aralkyl, heteroarylalkyl or heterocyclylalkyl;
   R₂ is alkyl, alkenyl, alkynyl, cycloalkyl, —NR_{g}Rₕ, aryl, aralkyl heteroaryl, heterocyclyl, heterocyclylalkyl or heteroarylalkyl, wherein
      R_{g} and Rₕ are independently selected from the group consisting of hydrogen, alkyl, alkenyl, alkynyl, cycloalkyl, aryl, aralkyl, heterocyclyl, heteroaryl, heterocyclylalkyl or heteroarylalkyl; or R_{g} and Rₕ join together to form a heterocyclyl ring;
   R₃ is alkyl, alkenyl, alkynyl, cycloalkyl, aryl, aralkyl, heteroarylalkyl or heterocyclylalkyl; and
   k is an integer from 1-4,
      and its pharmaceutically acceptable salts, pharmaceutically acceptable solvates, stereoisomers or polymorphs, wherein

In another aspect, provided are compounds selected from:
2-[6-(Benzylamino)-3-azabicyclo[3.1.0]hex-3-yl]-1-cyclopentyl-2-oxo-1-phenylethanol (Compound No. 1),
2-[6-(Benzylamino)-3-azabicyclo[3.1.0]hex-3-yl]-1-cyclohexyl-2-oxo-1-phenylethanol (Compound No. 2),
2-(6-Amino-3-azabicyclo[3.1.0]hex-3-yl)-1-cyclopentyl-1-(4-methylphenyl)-2-oxoethanol (Compound No. 3),
Tartarate salt of 2-[6-(allylamino)-3-azabicyclo[3.1.0]hex-3-yl]-1-cyclohexyl-2-oxo-1-phenylethanol (Compound No. 4),
Tartarate salt of 2-[6-(allylamino)-3-azabicyclo[3.1.0]hex-3-yl]-1-cyclohexyl-1-(4-methylphenyl)-2-oxo ethanol (Compound No. 5),
2-[6-(Allylamino)-3-azabicyclo[3.1.0]hex-3-yl]-1-cyclopentyl-1-(4-methylphenyl)-2-oxo ethanol (Compound No. 6),
2-[6-(Allylamino)-3-azabicyclo[3.1.0]hex-3-yl]-1-cyclopentyl-2-oxo-1-phenylethanol (Compound No. 7),
Tartarate salt of 2-[6-(allylamino)-3-azabicyclo[3.1.0]hex-3-yl]-1-cyclopentyl-1-(4-fluorophenyl)-2-oxoethanol (Compound No. 8),
Tartarate salt of 1-[6-(allylamino)-3-azabicyclo[3.1.0]hex-3-yl]-3-methyl-1-oxo-2-phenylbutan-2-ol (Compound No. 9),
1-Cyclopentyl-2-[6-(dimethylamino)-3-azabicyclo[3.1.0]hex-3-yl]-2-oxo-1-phenyl ethanol (Compound No. 10),
1-Cyclobutyl-2-oxo-1-phenyl-2-[6-(propylamino)-3-azabicyclo[3.1.0]hex-3-yl]ethanol (Compound No. 11),
1-Cyclohexyl-2-[6-(methylamino)-3-azabicyclo[3.1.0]hex-3-yl]-1-(4-methylphenyl)-2-oxoethanol (Compound No. 12),
1-Cyclohexyl-1-(4-fluorophenyl)-2-[6-(methylamino)-3-azabicyclo[3.1.0]hex-3-yl]-2-oxoethanol (Compound No. 13),
2-(4-Fluorophenyl)-3-methyl-1-[6-(methylamino)-3-azabicyclo[3.1.0]hex-3-yl]-1-oxo butan-2-ol (Compound No. 14),
1-Cyclopentyl-1-(4-methoxyphenyl)-2-[6-(methylamino)-3-azabicyclo[3.1.0]hex-3-yl]-2-oxoethanol (Compound No. 15),
1-Cyclohexyl-2-[6-(dimethylamino)-3-azabicyclo[3.1.0]hex-3-yl]-2-oxo-1-phenyl ethanol (Compound No. 16),
1-Cyclohexyl-2-{6-[(methylamino)methyl]-3-azabicyclo[3.1.0]hex-3-yl}-1-(4-methyl phenyl)-2-oxoethanol (Compound No. 17),
1-Cyclopentyl-2-[6-(dimethylamino)-3-azabicyclo[3.1.0]hex-3-yl]-2-oxo-1-(2-thienyl)ethanol (Compound No. 18),
1-Cyclohexyl-1-(4-fluorophenyl)-2-{6-[(methylamino)methyl]-3-azabicyclo[3.1.0] hex-3-yl}-2-oxoethanol (Compound No. 19),
2-(4-Fluorophenyl)-3-methyl-1-{6-[(methylamino)methyl]-3-azabicyclo[3.1.0]hex-3-yl}-1-oxobutan-2-ol (Compound No. 20),
2-(4-Fluorophenyl)-1-[6-(dimethylamino)-3-azabicyclo[3.1.0]hex-3-yl]-3-methyl-1-oxobutan-2-ol (Compound No. 21),
(1R)-1-Cyclopentyl-2-{6-[(methylamino)methyl]-3-azabicyclo[3.1.0]hex-3-yl}-2-oxo-1-phenylethanol (Compound No. 22),
Tartarate salt of (1R)-1-Cyclopentyl-2-[6-(methylamino)-3-azabicyclo[3.1.0]hex-3-yl]-2-oxo-1-phenylethanol (Compound No. 23),
2-[6-(Aminomethyl)-3-azabicyclo[3.1.0]hex-3-yl]-1-cyclopentyl-2-oxo-1-phenyl ethanol (Compound No. 24),
Tert-butyl ({3-[cyclopentyl(hydroxy)phenylacetyl]-3-azabicyclo[3.1.0]hex-6-yl}methyl)carbamate (Compound No. 25),
2-(6-Amino-3-azabicyclo[3.1.0]hex-3-yl)-1-cyclopentyl-2-oxo-1-phenylethanol (Compound No. 26),
Tert-butyl {3-[cyclopentyl(hydroxy)phenylacetyl]-3-azabicyclo[3.1.0]hex-6-yl} carbamate (Compound No. 27),
2-(6-Amino-3-azabicyclo[3.1.0]hex-3-yl)-2-oxo-1,1-diphenylethanol (Compound No. 28),
1-Cyclohexyl-2-[6-(hydroxymethyl)-3-azabicyclo[3.1.0]hex-3-yl]-2-oxo-1-phenyl ethanol (Compound No. 29),
1-Cyclohexyl-2-{6-[(ethylamino)methyl]-3-azabicyclo[3.1.0]hex-3-yl}-2-oxo-1-phenyl ethanol (Compound No. 30),
Tert-butyl {3-[cyclopentyl(hydroxy)phenylacetyl]-3-azabicyclo[3.1.0]hex-6-yl}methyl carbamate (Compound No. 31),
1-Cyclopentyl-2-[6-(methylamino)-3-azabicyclo[3.1.0]hex-3-yl]-2-oxo-1-phenyl ethanol (Compound No. 32),
Tert-butyl {3-[hydroxy(diphenyl)acetyl]-3-azabicyclo[3.1.0]hex-6-yl}methylcarbamate (Compound No. 33)
Tert-butyl [3-(2-hydroxy-2,2-diphenylethyl)-3-azabicyclo[3.1.0]hex-6-yl]carbamate (Compound No. 34),
2-(6-Amino-3-azabicyclo[3.1.0]hex-3-yl)-1,1-diphenylethanol (Compound No. 35),
2-(6-Amino-3-azabicyclo[3.1.0]hex-3-yl)-1-cyclopentyl-1-phenylethanol (Compound No. 36),
2-[6-(Aminomethyl)-3-azabicyclo[3.1.0]hex-3-yl]-1-cyclopentyl-1-phenylethanol (Compound No. 37),
2-{6-[(Methylamino)methyl]-3-azabicyclo[3.1.0]hex-3-yl}-2-oxo-1,1-diphenylethanol (Compound No. 38),
1-Cyclopentyl-2-{6-[(methylamino)methyl]-3-azabicyclo[3.1.0]hex-3-yl}-2-oxo-1-phenylethanol (Compound No. 39),
2-(6-Amino-3-azabicyclo[3.1.0]hex-3-yl)-1-cyclopentyl-2-oxo-1-(2-thienyl)ethanol (Compound No. 40),
2-(6-Amino-3-azabicyclo[3.1.0]hex-3-yl)-2-oxo-1-phenyl-1-(2-thienyl)ethanol (Compound No. 41),
1-Cyclohexyl-2-[6-(methylamino)-3-azabicyclo[3.1.0]hex-3-yl]-2-oxo-1-phenyl ethanol (Compound No. 42),
1-Cyclohexyl-2-{6-[(methylamino)methyl]-3-azabicyclo[3.1.0]hex-3-yl}-2-oxo-1-phenylethanol (Compound No. 43),
1-Cyclopentyl-2-[6-(isopropylamino)-3-azabicyclo[3.1.0]hex-3-yl]-2-oxo-1-phenyl ethanol (Compound No. 44),
1-Cyclohexyl-2-[6-(isopropylamino)-3-azabicyclo[3.1.0]hex-3-yl]-2-oxo-1-phenyl ethanol (Compound No. 45),
1,1,1-Trifluoro-3-[6-(methylamino)-3-azabicyclo[3.1.0]hex-3-yl]-3-oxo-2-phenylpropan-2-ol (Compound No. 46),
1,1,1-Trifluoro-3-{6-[(methylamino)methyl]-3-azabicyclo[3.1.0]hex-3-yl}-3-oxo-2-phenylpropan-2-ol (Compound No. 47),
1,1,1-Trifluoro-3-[6-(methylamino)-3-azabicyclo[3.1.0]hex-3-yl]-2-(4-methylphenyl)-3-oxopropan-2-ol (Compound No. 48),
1,1,1-Trifluoro-3-{6-[(methylamino)methyl]-3-azabicyclo[3.1.0]hex-3-yl}-2-(4-methyl phenyl)-3-oxopropan-2-ol (Compound No. 49),
(1R or 1S)-2-(6-Amino-3-azabicyclo[3.1.0]hex-3-yl)-1-cyclopentyl-2-oxo-1-(2-thienyl)ethanol (Compound No. 50),
   or their pharmaceutically acceptable salts, pharmaceutically acceptable solvates, stereoisomers or polymorphs.

In yet another aspect, provided are pharmaceutical compositions comprising one or more pharmaceutically acceptable carriers, excipients or diluents and a therapeutically effective amount of one or more compounds described herein, or pharmaceutically acceptable salts, pharmaceutically acceptable solvates, stereoisomers or polymorphs thereof.

In another aspect, provided are methods for treatment or prophylaxis of a disease or disorder of the respiratory, urinary or gastrointestinal systems, wherein the disease or disorder is mediated through muscarinic receptors, comprising administering to an animal or human in need thereof a therapeutically effective amount of one or more compounds described herein, or pharmaceutically acceptable salts, pharmaceutically acceptable solvates, stereoisomers or polymorphs thereof.

The method can include one or more of the following embodiments. For example, the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

In another aspect, provided are processes of preparing a compound of Formula IV, V, VI or VII, or a pharmaceutically acceptable salt, pharmaceutically acceptable solvate, stereoisomer or polymorph thereof, comprising the steps of:
a) condensing a compound of Formula II

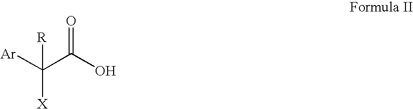

with a compound of Formula III (wherein p is 0 or 1, Rₖ is R_{y} or P)

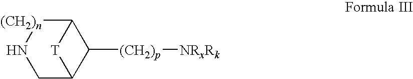

to form a compound of Formula IV;

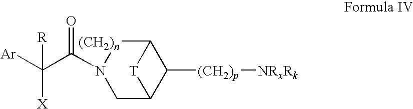

and
b) i) (Path a) optionally deprotecting the compound of Formula IV (wherein Rₖ is P) to form a compound of Formula V,

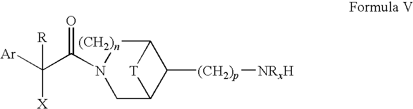

or
ii) (Path b)
   (A) optionally reducing the compound of Formula IV (wherein Rₓ is —(CH₂)_{q}CH═CH₂, wherein q is an integer from 1 to 3) to form a compound of Formula VI,

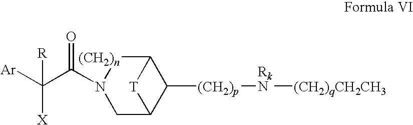

and
(B) optionally deprotecting the compound of Formula VI (wherein Rₖ is P) to form the compound of Formula VII,

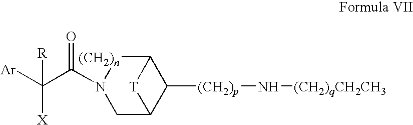

wherein,
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl, cycloalkyl or aryl;
T is —(CH₂)ₘ, —CH(Q)CH₂, —CH(Q), —CH₂—O—CH₂;
P is —C(═O)OC(CH₃)₃, —C(═O)C(CH₃)₂CHBr₂ or —C(═O)C(CH₃)₂CCl₃;
n is an integer from 0-2, wherein when n is zero then n represents a direct bond); and
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time.

The processes can include one or more of the following embodiments. For example, the compound of Formula II is condensed with a compound of Formula III to form a compound of Formula IV with one or more condensing agents, for example, 1-(3-dimethylaminopropyl)-3-ethyl carbodiimide hydrochloride, dicyclohexylcarbodiimide or mixtures thereof. In other embodiments, the compound of Formula II is condensed with a compound of Formula III to form a compound of Formula IV in the presence of one or more bases, for example, N-methylmorpholine, pyridine, triethylamine, diisopropylethylamine or mixtures thereof.

In other embodiments, the compound of Formula IV is deprotected to form a compound of Formula V in the presence of one or more acids or one or more supernucleophiles. The one or more acids are selected from hydrochloric acid, hydrobromic acid, trifluoroacetic acid or mixtures thereof. The one or more supernucleophiles are selected from lithium cobalt (I) phthalocyanine, zinc and acetic acid, cobalt phthalocyanine or mixtures thereof.

In other embodiments, the compound of Formula IV can be reduced to form compounds of Formula VI with one or more reducing agents, for example, hydrogen and palladium on carbon, sodium borohydride, sodium cyanoborohydride or mixtures thereof.

In yet other embodiments, the compound of Formula VI is deprotected to form the compound of Formula VII in the presence of one or more acids or one or more supernucleophiles. The one or more acids are selected from hydrochloric acid, hydrobromic acid, trifluoroacetic acid or mixtures thereof. The one or more supernucleophiles are selected from lithium cobalt (I) phthalocyanine, zinc and acetic acid, cobalt phthalocyanine or mixtures thereof.

In other embodiments, Rₖ is P and P is —C(═O)OC(CH₃)₃ and the one or more acids are selected from hydrochloric acid, trifluoroacetic acid or mixtures thereof;

Rₖ is P and P is —C(═O)OC(CH₃)₂CHBr₂ and the one or more acids are selected from hydrobromic acid, hydrochloric acid or mixtures thereof; or

Rₖ is P and P is —C(═O)OC(CH₃)₂CHCl₂ and the one or more supernucleophiles are selected from lithium cobalt (I) phthalocyanine, zinc and acetic acid, cobalt phthalocyanine or mixtures thereof.

In another aspect, provided are processes of preparing a compound of Formula IX, X or XII, or a pharmaceutically acceptable salt, pharmaceutically acceptable solvate, stereoisomer or polymorph thereof, comprising the steps of:
a) condensing a compound of Formula II

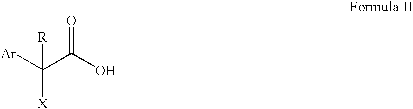

with a compound of Formula VIII

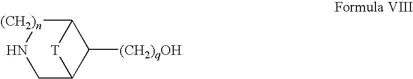

to form a compound of Formula IX;

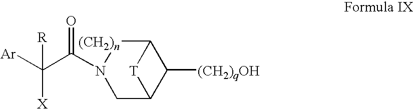

b) optionally O-derivatizing the compound of Formula IX to form a compound of Formula X (wherein P₁ is mesyl or tosyl),

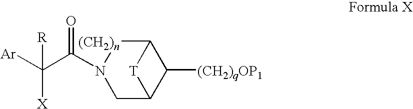

and
c) optionally reacting the compound of Formula X with a compound of Formula XI

HNRₓR_{y} Formula XI

to form a compound of Formula XII,

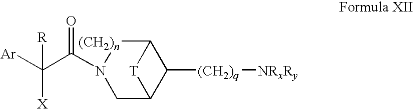

wherein,
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
T is —(CH₂)ₘ, —CH(Q)CH₂, —CH(Q), —CH₂—O—CH₂;
n is an integer from 0-2, wherein n represents a direct bond when n is zero;
q is an integer from 1 to 3; and
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time.

The processes can include one or more of the following embodiments. For example, the compound of Formula II is condensed with the compound of Formula VIII with one or more condensing agents, for example, 1-(3-dimethylaminopropyl)-3-ethyl carbodiimide hydrochloride, dicyclohexylcarbodiimide or mixtures thereof.

In other embodiments, the compound of Formula II is condensed with a compound of Formula III in the presence of one or more bases, for example, N-methylmorpholine, pyridine, triethylamine, diisopropylethylamine or mixtures thereof.

In yet other embodiments, the compound of Formula IX is O-derivatized in the presence of one or more bases, triethylamine, pyridine, N-methylmorpholine, diisopropylethylamine or mixtures thereof.

In another aspect, provided are processes of preparing a compound of Formula XIV or Formula XV, or a pharmaceutically acceptable salt, pharmaceutically acceptable solvate, stereoisomer or polymorph thereof, comprising the steps of:

a) reacting a compound of Formula XIII

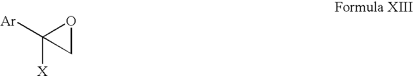

with a compound of Formula III

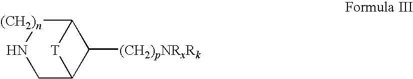

to form a compound of Formula XIV,

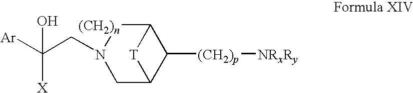

and
b) optionally deprotecting the compound of Formula XIV (wherein Rₖ is P) to form a compound of Formula XV,

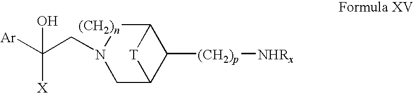

wherein
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
T is —(CH₂)ₘ, —CH(Q)CH₂, —CH(Q), —CH₂—O—CH₂;
n is an integer from 0-2, wherein n represents a direct bond when n is zero;
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time;
p is 0 or 1; and
P is —C(═O)OC(CH₃)₃, —C(═O)C(CH₃)₂CHBr₂ or —C(═O)C(CH₃)₂CCl₃).

The processes can include one or more of the following embodiments. For example, the compound of Formula XIII is reacted with the compound of Formula III in the presence of one or more bases, for example, potassium carbonate, sodium carbonate, lithium carbonate, potassium bicarbonate, sodium bicarbonate, lithium bicarbonate or mixtures thereof.

In other embodiments, the compound of Formula XIV is deprotected in the presence of one or more acids or one or more supernucleophiles. The one or more acids are selected from hydrochloric acid, hydrobromic acid, trifluoroacetic acid or mixtures thereof. The one or more supernucleophiles are selected from lithium cobalt (I) phthalocyanine, zinc and acetic acid, cobalt phthalocyanine or mixtures thereof.

In other embodiments, Rₖ is P and P is —C(═O)OC(CH₃)₃ and the one or more acids are selected from hydrochloric acid, trifluoroacetic acid or mixtures thereof;

Rₖ is P and P is —C(═O)OC(CH₃)₂CHBr₂ and the one or more acids are selected from hydrobromic acid, hydrochloric acid or mixtures thereof; or

Rₖ is P and P is —C(═O)OC(CH₃)₂CHCl₂ and the one or more supernucleophiles are selected from lithium cobalt (I) phthalocyanine, zinc and acetic acid, cobalt phthalocyanine or mixtures thereof.

### DETAILED DESCRIPTION OF THE INVENTION

In accordance with one aspect, provided herein are compounds having the structure of Formula I:

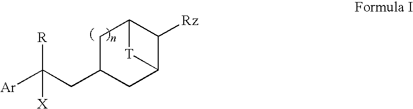

wherein
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl, cycloalkyl or aryl;
Y is —C(═O), —C(═S), —C(═Nacyl), —C(═N(NO₂)), —C(═CH(NO₂)), —C(═C(R₁)₂) or —CH₂—;
T is —(CH₂)ₘ, —CH(Q)CH₂, —CH(Q), —CH₂—O—CH₂;
Rz is hydrogen, hydroxy, alkoxy, hydroxyalkyl, aryloxy, —CHO, —CN, alkyl, alkenyl, alkynyl, cycloalkyl, carboxy, halogen, aryl, aralkyl, acyl, heteroaryl, heterocyclyl, heteroarylalkyl, heterocyclylalkyl, —(CH₂)ₖNRₓR_{y}, —SO₂R₂, —COOR₃, —C(═O)NRₓR_{y}, —NRₓR_{y}, —OC(═O)NRₓR_{y}, —NR₁C(═O)Rₓ or —NHC(═O)Rₓ; and
n is an integer from 0-2, wherein when n is zero then n represents a direct bond);
   wherein
   Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time);
   R₁ is hydrogen, alkyl, alkenyl, alkynyl, cycloalkyl, aryl, heterocyclyl, heteroaryl, aralkyl, heteroarylalkyl or heterocyclylalkyl;
   m is an integer from 0-3, wherein T represents a direct bond when m is zero;
   Q is alkyl, alkenyl, alkynyl, cycloalkyl, aryl, heteroaryl, heterocyclyl, aralkyl, heteroarylalkyl or heterocyclylalkyl;
   R₂ is alkyl, alkenyl, alkynyl, cycloalkyl, —NR_{g}Rₕ, aryl, aralkyl, heteroaryl, heterocyclyl, heterocyclylalkyl or heteroarylalkyl, wherein
      R_{g} and Rₕ are independently selected from the group consisting of hydrogen, alkyl, alkenyl, alkynyl, cycloalkyl, aryl, aralkyl, heterocyclyl, heteroaryl, heterocyclylalkyl or heteroarylalkyl; or R_{g} and Rₕ join together to form a heterocyclyl ring;
   R₃ is alkyl, alkenyl, alkynyl, cycloalkyl, aryl, aralkyl, heteroarylalkyl or heterocyclylalkyl; and
   k is an integer from 1-4.

In accordance with another aspect, provided are methods for the treatment or prophylaxis of a disease or disorder of the respiratory, urinary or gastrointestinal system in an animal or a human suffering therefrom, wherein the disease or disorder is mediated through muscarinic receptors. The methods include administration of at least one compound described herein to an animal or human in need thereof.

Diseases or disorders of the respiratory system include, for example, bronchial asthma, chronic obstructive pulmonary disorders (COPD), pulmonary fibrosis, and the like. Diseases or disorders of the urinary system include, for example, urinary incontinence, lower urinary tract symptoms (LUTS), and the like. Diseases or disorders of the gastrointestinal system include, for example, irritable bowel syndrome, obesity, diabetes or gastrointestinal hyperkinesis.

In accordance with another aspect, provided are methods for the treatment or prophylaxis of a disease or disorder associated with muscarinic receptors comprising administering to a patient in need thereof an effective amount of one or more compounds described herein.

The compounds described herein exhibit significant potency in terms of their activity, as determined by in vitro receptor binding and functional assays and in vivo experiments using anaesthetized rabbits. The compounds that were found active in vitro were tested in vivo. Some of the compounds are potent muscarinic receptor antagonists with high affinity towards M₂ and/or M₃ receptors with M₅ sparing activity. Therefore, pharmaceutical compositions for the possible treatment for the disease or disorders associated with muscarinic receptors are provided. In addition, the compounds can be administered by any route of administration, including, for example, orally or parenterally.

The following definitions apply to terms as used herein.

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NRₐ—, wherein Rₐ can be hydrogen, alkyl, alkenyl, alkynyl cycloalkyl or aryl. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further (referred herein as “substituted alkyl”) with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)Rₚ, —NRₚR_{q}, —C(═O)NRₚR_{q}, —NHC(═O)NRₚR_{q}, —C(═O)heteroaryl, C(═O)heterocyclyl, —O—C(═O)NRₚR_{q} {wherein Rₚ and R_{q} are independently selected from hydrogen, hydroxy, alkyl, alkenyl, alkynyl, cycloalkyl, alkoxy, cycloalkenyl, aryl, aralkyl, heterocyclyl, heteroaryl, heterocyclylalkyl, heteroarylalkyl, or carboxy}, nitro, hydroxyamino, alkoxyamino or S(O)ₘR₆₆ (wherein m is an integer from 0-2 and R₆₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NRₚR_{q}, —C(═O)NRₚR_{q}, —OC(═O)NRₚR_{q}, —NHC(═O)NRₚR_{q} (wherein Rₚ and R_{q} are the same as defined earlier), hydroxy, alkoxy, halogen, CF₃, cyano, and S(O)ₘR₆₆ (wherein m is an integer from 0-2 and R₆₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)ORₚ (wherein Rₚ is the same as defined earlier), S(O)ₘR₆₆ (wherein m is an integer from 0-2 and R₆₆ is as defined earlier), or —C(═O)NRₚR_{q} (wherein Rₚ and R_{q} are as defined earlier)}. Unless otherwise constrained by the definition, all substituents may be substituted further by 1-3 substituents selected from alkyl, carboxy, carboxyalkyl, —NRₚR_{q}, —C(═O)NRₚR_{q}, —O—C(═O)NRₚR_{q} (wherein Rₚ and R_{q} are the same as defined earlier) hydroxy, alkoxy, halogen, CF₃, cyano, and S(O)ₘR₆₆ (wherein m is an integer from 0-2 and R₆₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. It can be optionally interrupted by atom(s) or group(s) independently chosen from oxygen, sulfur, phenylene, sulphinyl, sulphonyl and —NRₐ—, wherein Rₐ can be hydrogen, alkyl, alkenyl, alkynyl, cycloalkyl or aryl. In the event that alkenyl is attached to a heteroatom, the double bond cannot be alpha to the heteroatom. Alkenyl groups may be substituted further (referred to herein as “substituted alkenyl”) with one or more substituents selected from alkyl, alkenyl, alkynyl, alkoxy, cycloalkyl, cycloalkenyl, acyl, acylamino, acyloxy, —NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro, or SO₂R₆₆ (wherein R₆₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NRₚR_{q}, —C(═O)NRₚR_{q}, —O—C(═O)NRₚR_{q} (wherein Rₚ and R_{q} are the same as defined earlier) and —SO₂R₆₆ (wherein R₆₆ is same as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. It can be optionally interrupted by atom(s) or group(s) independently chosen from oxygen, sulfur, phenylene, sulphinyl, sulphonyl and —NRₐ—, wherein Rₐ can be hydrogen, alkyl, alkenyl, alkynyl, cycloalkyl or aryl. In the event that alkynyl is attached to a heteroatom, the triple bond cannot be alpha to the heteroatom. Alkynyl groups may be substituted further (referred to herein as “substituted alkynyl”) with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)Rₚ, —NRₚR_{q}, —NHC(═O)NRₚR_{q}, —C(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NRₚR_{q}, —C(═O)NRₚR_{q}, —NHC(═O)NRₚR_{q}, —C(═O)NRₚR_{q} (wherein Rₚ and R_{q} are the same as defined earlier), cyano, or S(O)ₘR₆₆ (wherein m is an integer from 0-2 and R<56 is same as defined earlier). Groups such as ethynyl, (—C≡CH), propargyl (or propynyl, —CH₂C≡CH), and the like exemplify this term.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo [2.2.1] heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NRₚR_{q}, —NHC(═O)NRₚR_{q}, —NHC(═O)Rₚ, —C(═O)NRₚR_{q}, —O—C(═O)NRₚR_{q} (wherein Rₚ and R_{q} are the same as defined earlier), nitro, heterocyclyl, heteroaryl, heterocyclylalkyl, heteroarylalkyl, or S(O)ₘR₆₆ (wherein m is an integer from 0-2 and R₆₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NRₚR_{q}, —C(═O)NRₚR_{q}, —NHC(═O)NRₚR_{q}, —O—C(═O)NRₚR_{q} (wherein Rₚ and R_{q} are the same as defined earlier), cyano or S(O)ₘR₆₆ (wherein m is an integer from 0-2 and R₆₆ is same as defined earlier).

The term “alkoxy” denotes the group O-alkyl, wherein alkyl is the same as defined above.

The term “aryl” herein refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₛ (wherein R₅ is hydrogen, alkyl, alkenyl, alkynyl, cycloalkyl, aralkyl, heterocyclylalkyl, heteroarylalkyl), NHC(═O)Rₚ, —NRₚR_{q}, —C(═O)NRₚR_{q}, —NHC(═O)NRₚR_{q}, —O—C(═O)NRₚR_{q}, S(O)ₘR₆₆ (wherein m is an integer from 0-2 and R₆₆ is same as defined earlier), carboxy, optionally substituted heterocyclyl, heteroaryl, heterocyclylalkyl, heteroarylalkyl, amino carbonyl amino, mercapto, haloalkyl, optionally substituted aryl, optionally substituted heterocyclylalkyl, thioalkyl, —CONHRₚ, —OCORₚ, —CORₚ, —NHSO₂Rₚ, or —SO₂NHRₚ (wherein Rₚ and R_{q} are the same as defined earlier). The aryl group optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “carboxy” as defined herein refers to —C(═O)OH.

The term “heteroaryl,” unless otherwise specified, refers to an aromatic ring structure containing 5 or 6 ring atoms, or a bicyclic or tricyclic aromatic group having from 8 to 14 ring atoms, with one or more heteroatom(s) independently selected from N, O or S. Heteroaryl groups can be optionally substituted with 1 to 4 substituent(s) (referred herein as “substituted heteroaryl”) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NRₚR_{q}, CH═NOH, —(CH₂)_{w}C(═O)Rₜ {wherein w is an integer from 0-4 and Rₜ is hydrogen, hydroxy, ORₚ, NRₚR_{q}, —NHOR_{z} or —NHOH}, —C(═O)NRₚR_{q} and —NHC(═O)NRₚR_{q}, S(O)ₘR₆₆, —O—C(═O)NRₚR_{q}, —O—C(═O)Rₚ, —O—C(═O)ORₚ (wherein m, R₆₆, Rₚ and R_{q} are as defined earlier, and R₂ is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, benzthiazinyl, benzthiazinonyl, benzoxazinyl, benzoxazinonyl, quinazolyl, carbazolyl phenothiazinyl, phenoxazinyl and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)Rₚ, —O—C(═O)ORₚ>—C(═O)NRₚR_{q}, S(O)ₘR₆₆, —O—C(═O)NRₚR_{q}, —NHC(═O)NRₚR_{q}, —NRₚR_{q}, NRₚR_{q}, mercapto, haloalkyl, thioalkyl, —COORₚ, —COONHRₚ, —CORₚ, —NHSO₂Rₚ, SO₂NHRₚ (wherein m, R₆₆, Rₚ and R_{q} are as defined earlier) or guanidine. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxozinyl, benzothiazinyl, imidazolyl, benzimidazolyl, tetrazolyl, carbazolyl, indolyl, phenoxozinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine, and the like.

“Heteroarylalkyl” refers to heteroaryl (wherein heteroaryl is same as defined earlier) linked through alkyl (wherein alkyl is the same as defined above) portion and the said alkyl portion contains carbon atoms from 1-6.

“Heterocyclylalkyl” refers to heterocyclyl (wherein heterocyclyl is same as defined earlier) linked through alkyl (wherein alkyl is the same as defined above) portion and the said alkyl portion contains carbon atoms from 1-6.

“Acyl” refers to —C(═O)R″ wherein R″ is selected from the group consisting of hydrogen, alkyl, cycloalkyl, aryl, aralkyl, heteroaryl, heterocyclyl, heteroarylalkyl or heterocyclylalkyl.

The term “leaving group” generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “Protecting groups” is used herein to refer to known moieties which have the desirable property of preventing specific chemical reaction at a site on the molecule undergoing chemical modification intended to be left unaffected by the particular chemical modification. Also the term protecting group, unless or other specified may be used with groups such as hydroxy, amino, carboxy and example of such groups are found in T. W. Greene and P. G. M. Wuts, “Protective Groups in Organic synthesis”, 2^{nd} Edn. John Wiley and Sons, New York, N.Y., which is incorporated herein by reference. The species of the carboxylic protecting groups, amino protecting groups or hydroxy protecting group employed is not so critical so long as the derivatized moiety/moieties is/are stable to conditions of subsequent reactions and can be removed at the appropriate point without disrupting the remainder of the molecule.

The term “pharmaceutically acceptable salts” of the compounds represented by the Formula I were prepared so as to solubilize the compound in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, toluenesulphonate and methanesulphonate). These salts may be prepared by various techniques, such as treating the compound with an equivalent amount of inorganic or organic, acid or base in a suitable solvent.

The salt forms differ from compounds described herein in certain physical properties, such as solubility, but the salts are otherwise equivalent for the purpose of this invention.

The compounds of the present invention may be prepared by techniques well known in the art and familiar to a practitioner skilled in art of this invention. In addition, the compounds of the present invention may be prepared by the process described herein, this process is not the only means by which the compounds described may be synthesized. Further, the various synthetic steps described herein may be performed in an alternate sequence in order to give the desired compounds.

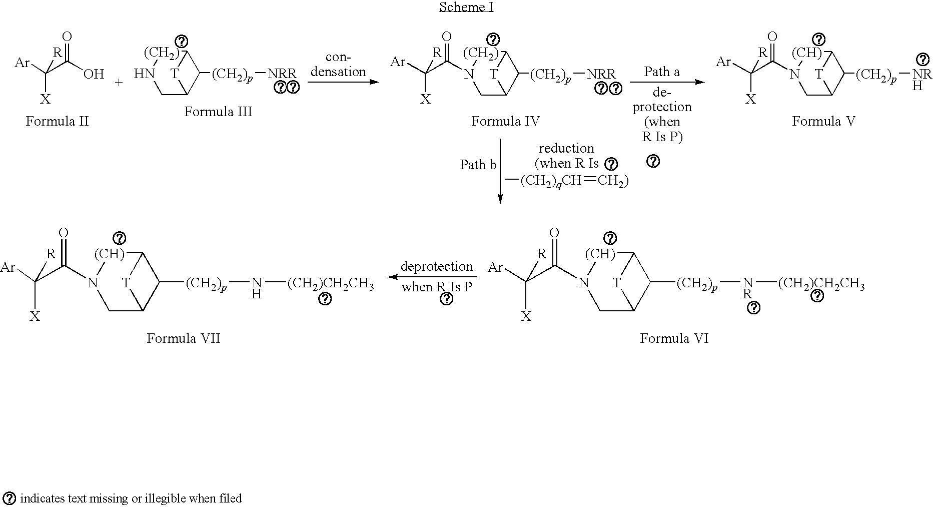

The compounds of Formulae IV, V, VI and VII can be prepared, for example, by the reaction sequence in Scheme I. Thus compounds of Formula III (wherein R and Ar are the same as defined earlier) can be condensed with compounds of Formula III (wherein p is 0 or 1, Rₖ is R_{y} or P (wherein P is —C(═O)OC(CH₃)₃, —C(═O)C(CH₃)₂CHBr₂ or —C(═O)C(CH₃)₂CCl₃) and n, T, Rₓ and R_{y} the same as defined earlier) to form compounds of Formula IV. Compounds of Formula IV can be deprotected (Path a) (when Rₖ is P) to form compounds of Formula V; or compounds of Formula IV (Path b) can be reduced (when Rₓ is —(CH₂)_{q}CH═CH₂ (wherein q is the same as defined earlier)) to form compounds of Formula VI. Compounds of Formula VI can be deprotected (when Rₖ is P) to form compounds of Formula VII.

Compounds of Formula II can be condensed with compounds of Formula III to form compounds of Formula IV with one or more condensing agents, for example, carbodiimide compounds, e.g., 1-(3-dimethylaminopropyl)-3-ethyl carbodiimide hydrochloride, dicyclohexylcarbodiimide or mixtures thereof. The condensation reaction can also be carried out in the presence of one or more bases, for example, morpholines, pyridines, amines or mixtures thereof, e.g., N-methylmorpholine, pyridine, triethylamine, diisopropylethylamine or mixtures thereof. The condensation reaction can also be carried out in one or more organic solvents, for example dimethyl formamide, tetrahydrofuran, dioxane, diethylether or mixtures thereof.

Compounds of Formula IV can be deprotected (Path a) to form compounds of Formula V in the presence of one or more acids or one or more supernucleophiles. Suitable acids include inorganic or organic acids including, for example, hydrochloric acid, hydrobromic acid, trifluoroacetic acid or mixtures thereof. Suitable supernucleophiles include, for example, lithium cobalt (I) phthalocyanine, zinc and acetic acid, cobalt phthalocyanine or mixtures thereof. The deprotection reaction can also be carried out in one or more organic solvents, for example, alcohols, e.g., methanol, ethanol, propanol, isopropylalcohol or mixtures thereof.

In one embodiment, compounds of Formula IV, wherein Rₖ is P and P is —C(═O)OC(CH₃)₃, can be deprotected in, for example, hydrochloric acid in methanol or ethanol or trifluoroacetic acid. In another embodiment, compounds of Formula IV, wherein Rₖ is P and P is —C(═O)C(CH₃)₂CHBr₂, can be deprotected in hydrobromic acid, hydrochloric acid or mixtures thereof. In another embodiment, compounds of Formula IV, wherein Rₖ is P and P is —C(═O)OC(CH₃)₂CCl₃, can be deprotected in the presence of one or more of lithium cobalt (I) phthalocyanine, zinc and acetic acid, cobalt phthalocyanine or mixtures thereof.

Compounds of Formula IV (Path b) (when Rₓ is —(CH₂)_{q}CH═CH₂)) can be reduced to form compounds of Formula VI with one or more reducing agents, for example, hydrogen and palladium on carbon, sodium borohydride, sodium cyanoborohydride or mixtures thereof. The reduction reaction can also be carried out in one or more organic solvents, for example, alcohols, e.g., methanol, ethanol, propanol, isopropylalcohol or mixtures thereof.

Compounds of Formula VI (when Rₖ is P wherein P is the same as defined above) can be deprotected following the procedure as described for the synthesis of compound of Formula V from a compound of Formula IV. In particular, compounds of Formula VI can be deprotected in the presence of one or more acids or one or more supernucleophiles. Suitable acids include inorganic or organic acids including, for example, hydrochloric acid, hydrobromic acid, trifluoroacetic acid or mixtures thereof. Suitable supernucleophiles include, for example, lithium cobalt (I) phthalocyanine, zinc and acetic acid, cobalt phthalocyanine or mixtures thereof. The deprotection reaction can also be carried out in one or more organic solvents, for example, alcohols, e.g., methanol, ethanol, propanol, isopropylalcohol or mixtures thereof.

In one embodiment, compounds of Formula VI, wherein Rₖ is P and P is —C(═O)OC(CH₃)₃, can be deprotected in, for example, hydrochloric acid in methanol or ethanol or trifluoroacetic acid. In another embodiment, compounds of Formula VI, wherein Rₖ is P and P is —C(═O)C(CH₃)₂CHBr₂, can be deprotected in hydrobromic acid, hydrochloric acid or mixtures thereof. In another embodiment, compounds of Formula VI, wherein Rₖ is P and P is —C(═O)OC(CH₃)₂CCl₃, can be deprotected in the presence of one or more of lithium cobalt (I) phthalocyanine, zinc and acetic acid, cobalt phthalocyanine or mixtures thereof.

Compounds prepared following Scheme I include, for example:
2-[6-(Benzylamino)-3-azabicyclo[3.1.0]hex-3-yl]-1-cyclopentyl-2-oxo-1-phenylethanol (Compound No. 1),
2-[6-(Benzylamino)-3-azabicyclo[3.1.0]hex-3-yl]-1-cyclohexyl-2-oxo-1-phenylethanol (Compound No. 2),
2-(6-Amino-3-azabicyclo[3.1.0]hex-3-yl)-1-cyclopentyl-1-(4-methylphenyl)-2-oxoethanol (Compound No. 3),
Tartarate salt of 2-[6-(allylamino)-3-azabicyclo[3.1.0]hex-3-yl]-1-cyclohexyl-2-oxo-1-phenylethanol (Compound No. 4),
Tartarate salt of 2-[6-(allylamino)-3-azabicyclo[3.1.0]hex-3-yl]-1-cyclohexyl-1-(4-methylphenyl)-2-oxo ethanol (Compound No. 5),
2-[6-(Allylamino)-3-azabicyclo[3.1.0]hex-3-yl]-1-cyclopentyl-1-(4-methylphenyl)-2-oxo ethanol (Compound No. 6),
2-[6-(Allylamino)-3-azabicyclo[3.1.0]hex-3-yl]-1-cyclopentyl-2-oxo-1-phenylethanol (Compound No. 7),
Tartarate salt of 2-[6-(allylamino)-3-azabicyclo[3.1.0]hex-3-yl]-1-cyclopentyl-1-(4-fluorophenyl)-2-oxoethanol (Compound No. 8),
Tartarate salt of 1-[6-(allylamino)-3-azabicyclo[3.1.0]hex-3-yl]-3-methyl-1-oxo-2-phenylbutan-2-ol (Compound No. 9),
1-Cyclopentyl-2-[6-(dimethylamino)-3-azabicyclo[3.1.0]hex-3-yl]-2-oxo-1-phenyl ethanol (Compound No. 10),
1-Cyclobutyl-2-oxo-1-phenyl-2-[6-(propylamino)-3-azabicyclo[3.1.0]hex-3-yl]ethanol (Compound No. 11),
1-Cyclohexyl-2-[6-(methylamino)-3-azabicyclo[3.1.0]hex-3-yl]-1-(4-methylphenyl)-2-oxoethanol (Compound No. 12),
1-Cyclohexyl-1-(4-fluorophenyl)-2-[6-(methylamino)-3-azabicyclo[3.1.0]hex-3-yl]-2-oxoethanol (Compound No. 13),
2-(4-Fluorophenyl)-3-methyl-1-[6-(methylamino)-3-azabicyclo[3.1.0]hex-3-yl]-1-oxo butan-2-ol (Compound No. 14),
1-Cyclopentyl-1-(4-methoxyphenyl)-2-[6-(methylamino)-3-azabicyclo[3.1.0]hex-3-yl]-2-oxoethanol (Compound No. 15),
1-Cyclohexyl-2-[6-(dimethylamino)-3-azabicyclo[3.1.0]hex-3-yl]-2-oxo-1-phenyl ethanol (Compound No. 16),
1-Cyclohexyl-2-{6-[(methylamino)methyl]-3-azabicyclo[3.1.0]hex-3-yl}-1-(4-methyl phenyl)-2-oxoethanol (Compound No. 17),
1-Cyclopentyl-2-[6-(dimethylamino)-3-azabicyclo[3.1.0]hex-3-yl]-2-oxo-1-(2-thienyl)ethanol (Compound No. 18),
1-Cyclohexyl-1-(4-fluorophenyl)-2-{6-[(methylamino)methyl]-3-azabicyclo[3.1.0] hex-3-yl}-2-oxoethanol (Compound No. 19),
2-(4-Fluorophenyl)-3-methyl-1-{6-[(methylamino)methyl]-3-azabicyclo[3.1.0]hex-3-yl}-1-oxobutan-2-ol (Compound No. 20),
2-(4-Fluorophenyl)-1-[6-(dimethylamino)-3-azabicyclo[3.1.0]hex-3-yl]-3-methyl-1-oxobutan-2-ol (Compound No. 21),
(1R)-1-Cyclopentyl-2-{6-[(methylamino)methyl]-3-azabicyclo[3.1.0]hex-3-yl}-2-oxo-1-phenylethanol (Compound No. 22),
Tartarate salt of (1R)-1-Cyclopentyl-2-[6-(methylamino)-3-azabicyclo[3.1.0]hex-3-yl]-2-oxo-1-phenylethanol (Compound No. 23),
2-[6-(Aminomethyl)-3-azabicyclo[3.1.0]hex-3-yl]-1-cyclopentyl-2-oxo-1-phenyl ethanol (Compound No. 24),
Tert-butyl {3-[cyclopentyl(hydroxy)phenylacetyl]-3-azabicyclo[3.1.0]hex-6-yl}methyl)carbamate (Compound No. 25),
2-(6-Amino-3-azabicyclo[3.1.0]hex-3-yl)-1-cyclopentyl-2-oxo-1-phenylethanol (Compound No. 26),
Tert-butyl {3-[cyclopentyl(hydroxy)phenylacetyl]-3-azabicyclo[3.1.0]hex-6-yl} carbamate (Compound No. 27),
2-(6-Amino-3-azabicyclo[3.1.0]hex-3-yl)-2-oxo-1,1-diphenylethanol (Compound No. 28),
Tert-butyl {3-[cyclopentyl(hydroxy)phenylacetyl]-3-azabicyclo[3.1.0]hex-6-yl}methyl carbamate (Compound No. 31),
1-Cyclopentyl-2-[6-(methylamino)-3-azabicyclo[3.1.0]hex-3-yl]-2-oxo-1-phenyl ethanol (Compound No. 32),
Tert-butyl {3-[hydroxy(diphenyl)acetyl]-3-azabicyclo[3.1.0]hex-6-yl}methylcarbamate (Compound No. 33)
2-{6-[(Methylamino)methyl]-3-azabicyclo[3.1.0]hex-3-yl}-2-oxo-1,1-diphenylethanol (Compound No. 38),
1-Cyclopentyl-2-{6-[(methylamino)methyl]-3-azabicyclo[3.1.0]hex-3-yl}-2-oxo-1-phenylethanol (Compound No. 39),
2-(6-Amino-3-azabicyclo[3.1.0]hex-3-yl)-1-cyclopentyl-2-oxo-1-(2-thienyl)ethanol (Compound No. 40),
2-(6-Amino-3-azabicyclo[3.1.0]hex-3-yl)-2-oxo-1-phenyl-1-(2-thienyl)ethanol (Compound No. 41),
1-Cyclohexyl-2-[6-(methylamino)-3-azabicyclo[3.1.0]hex-3-yl]-2-oxo-1-phenyl ethanol (Compound No. 42),
1-Cyclohexyl-2-{6-[(methylamino)methyl]-3-azabicyclo[3.1.0]hex-3-yl}-2-oxo-1-phenylethanol (Compound No. 43),
1-Cyclopentyl-2-[6-(isopropylamino)-3-azabicyclo[3.1.0]hex-3-yl]-2-oxo-1-phenyl ethanol (Compound No. 44),
1-Cyclohexyl-2-[6-(isopropylamino)-3-azabicyclo[3.1.0]hex-3-yl]-2-oxo-1-phenyl ethanol (Compound No. 45),
1,1,1-Trifluoro-3-[6-(methylamino)-3-azabicyclo[3.1.0]hex-3-yl]-3-oxo-2-phenyl propan-2-ol (Compound No. 46),
1,1,1-Trifluoro-3-{6-[(methylamino)methyl]-3-azabicyclo[3.1.0]hex-3-yl}-3-oxo-2-phenylpropan-2-ol (Compound No. 47),
1,1,1-Trifluoro-3-[6-(methylamino)-3-azabicyclo[3.1.0]hex-3-yl]-2-(4-methylphenyl)-3-oxopropan-2-ol (Compound No. 48),
1,1,1-Trifluoro-3-{6-[(methylamino)methyl]-3-azabicyclo[3.1.0]hex-3-yl}-2-(4-methyl phenyl)-3-oxopropan-2-ol (Compound No. 49),
(1R or 1S)-2-(6-Amino-3-azabicyclo[3.1.0]hex-3-yl)-1-cyclopentyl-2-oxo-1-(2-thienyl)ethanol (Compound No. 50).

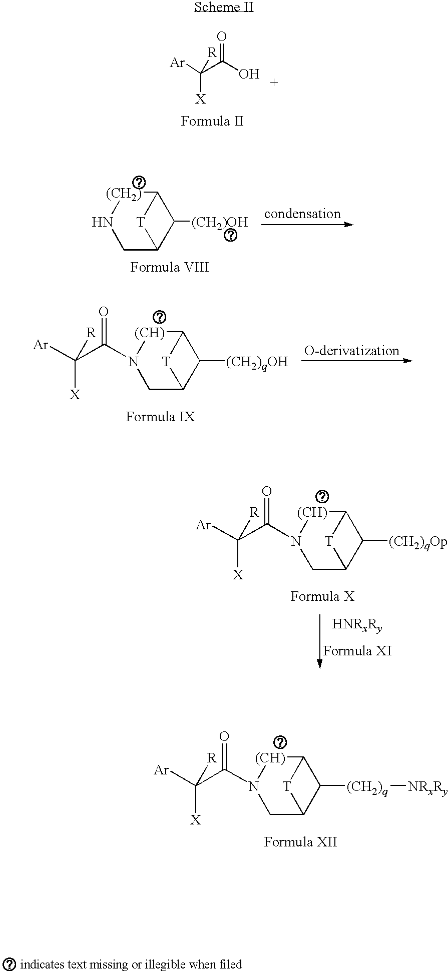

The compounds of Formulae IX, X and XII can be prepared, for example, by the reaction sequence in Scheme n. Thus compounds of Formula II (wherein Ar and X are the same as defined earlier) can be condensed with a compound of Formula VIII (wherein n and T are the same as defined earlier and q is an integer from 1 to 3) to form compounds of Formula IX. Compounds of Formula IX can be O-derivatized to form compounds of Formula X (wherein P₁ is mesyl or tosyl). Compounds of Formula X can be reacted with compounds of Formula XI (wherein Rₓ and R_{y} are the same as defined earlier) to form compounds of Formula XII.

Compounds of Formula n can be condensed with compounds of Formula VIII to form compounds of Formula IX with one or more condensing agents, for example, carbodiimides, e.g., 1-(3-dimethylaminopropyl)-3-ethyl-carbodiimide hydrochloride, dicyclohexylcarbodiimide or mixtures thereof. The condensation reaction can also be carried out in the presence of one or more bases, for example, morpholines, pyridines, amines or mixtures thereof, e.g., N-methylmorpholine, pyridine, triethylamine, diisopropylethylamine or mixtures thereof. The condensation reactions can also be carried out in one or more organic solvents, for example, dimethylformamide, tetrahydrofuran, diethylether, dioxane or mixtures thereof.

Compounds of Formula IX can be O-derivatized to form compounds of Formula X in the presence of one or more bases, for example, morpholines, pyridines, amines or mixtures thereof, e.g., triethylamine, pyridine, N-methylmorpholine, diisopropylethylamine or mixtures thereof. The O-derivatization can also be carried out in one or more organic solvents, for example, dichloromethane, dichloroethane, chloroform, carbon tetrachloride or mixtures thereof.

Compounds of Formula X can be reacted with compounds of Formula XI to form compounds of Formula XII in one or more organic solvents, for example, alcohol, e.g., ethanol, methanol, propanol, isopropyl alcohol or mixtures thereof.

Compounds(s) prepared following Scheme II include, for example:
1-Cyclohexyl-2-[6-(hydroxymethyl)-3-azabicyclo[3.1.0]hex-3-yl]-2-oxo-1-phenyl ethanol (Compound No. 29),
1-Cyclohexyl-2-{6-[(ethylamino)methyl]-3-azabicyclo[3.1.0]hex-3-yl}-2-oxo-1-phenyl ethanol (Compound No. 30).

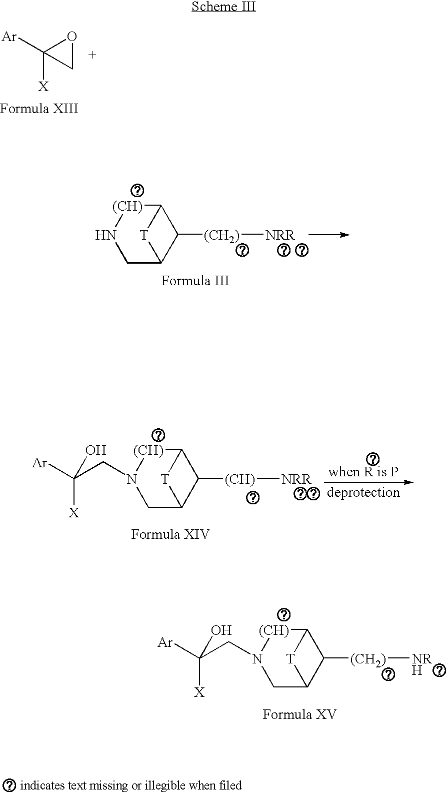

The compounds of Formulae XIV and Formula XV can be prepared, for example by the reaction sequence in Scheme III Thus compounds of Formula XIII (wherein Ar and X are the same as defined earlier) can be reacted with compounds of Formula III (wherein n, T, p, Rₓ and Rₖ are the same as defined earlier) to form compounds of Formula XIV. Compounds of Formula XIV can be deprotected (when Rₖ is P (wherein P is the same as defined earlier)) to form compounds of Formula XV.

Compounds of Formula XIII can be reacted with compounds of Formula III to form compounds of Formula XIV in the presence of one or more bases, for example, Group I carbonates, Group I bicarbonates, Group II carbonates, Group II bicarbonates, or mixtures thereof, e.g., potassium carbonate, sodium carbonate, lithium carbonate, potassium bicarbonate, sodium bicarbonate, lithium bicarbonate or mixtures thereof. The reaction can also be carried out in one or more organic solvents, for example, dimethylformamide, tetrahydrofuran, dioxane, diethylether or mixtures thereof.

The deprotection of a compound of Formula XIV (when Rₖ is P and P is —C(═O)OC(CH₃)₃)—C(═O)OC(CH₃)₂CHBr₂, —C(═O)OC(CH₃)₂CCl₃) to form compounds of Formula XV can be carried out by following the procedure as described in preparing compounds of Formula V from compounds of Formula IV in Scheme I above. In particular, the deprotection of compounds of Formula XIV can be carried out in the presence of one or more acids or one or more supernucleophiles. Suitable acids include inorganic or organic acids including, for example, hydrochloric acid, hydrobromic acid, trifluoroacetic acid or mixtures thereof. Suitable supernucleophiles include, for example, lithium cobalt (I) phthalocyanine, zinc and acetic acid, cobalt phthalocyanine or mixtures thereof. The deprotection reaction can also be carried out in one or more organic solvents, for example, alcohols, e.g., methanol, ethanol, propanol, isopropylalcohol or mixtures thereof.

In one embodiment, compounds of Formula XIV, wherein Rₖ is P and P is —C(═O)OC(CH₃)₃, can be deprotected in, for example, methanolic hydrochloric acid or ethanol or trifluoroacetic acid. In another embodiment, compounds of Formula XIV, wherein Rₖ is P and P is —C(═O)C(CH₃)₂CHBr₂, can be deprotected in hydrobromic acid, hydrochloric acid or mixtures thereof. In another embodiment, compounds of Formula XIV, wherein Rₖ is P and P is —C(═O)OC(CH₃)₂CCl₃, can be deprotected in the presence of one or more of lithium cobalt (I) phthalocyanine, zinc and acetic acid, cobalt phthalocyanine or mixtures thereof.

Compound(s) prepared following Scheme III include, for example:
Tert-butyl [3-(2-hydroxy-2,2-diphenylethyl)-3-azabicyclo[3.1.0]hex-6-yl]carbamate (Compound No. 34),
2-(6-Amino-3-azabicyclo[3.1.0]hex-3-yl)-1,1-diphenylethanol (Compound No. 35),
2-(6-Amino-3-azabicyclo[3.1.0]hex-3-yl)-1-cyclopentyl-1-phenylethanol (Compound No. 36),
2-[6-(Aminomethyl)-3-azabicyclo[3.1.0]hex-3-yl]-1-cyclopentyl-1-phenylethanol (Compound No. 37).

In the above scheme, where specific bases, condensing agents, protecting groups, deprotecting agents, solvents, catalysts, temperatures, etc. are mentioned, it is to be understood that other bases, condensing agents, protecting groups, deprotecting agents, solvents, catalysts, temperatures, etc. known to those skilled in the art may be used. Similarly, the reaction temperature and duration may be adjusted according to the desired needs.

Exemplary compounds described herein that can be formed by the schemes described above are listed in the Table below:

| Cpd | |
|---|---|
| No. | Structure |
| 1 | 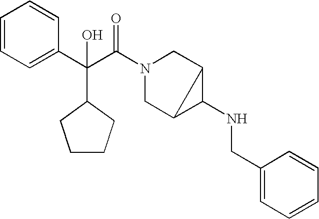 |
| 2. | 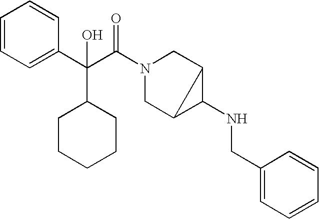 |
| 3. | 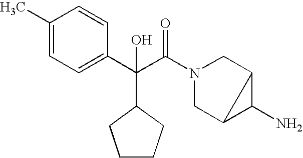 |
| 4. | 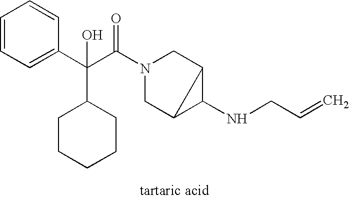 |
| 5. | 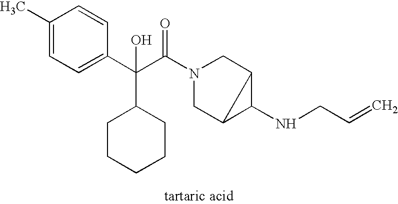 |
| 6. | 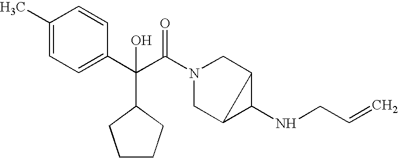 |
| 7. | 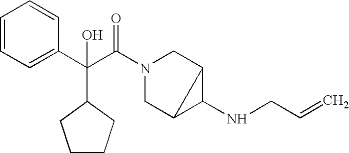 |
| 8. | 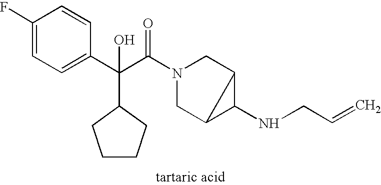 |
| 9. | 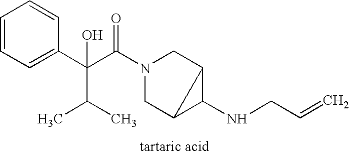 |
| 10. | 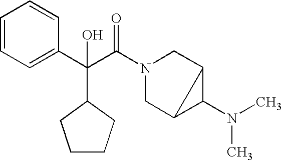 |
| 11. | 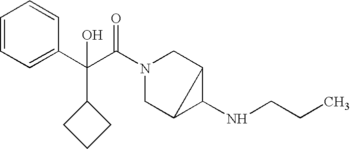 |
| 12. | 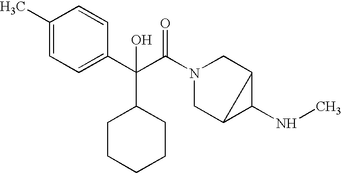 |
| 13. | 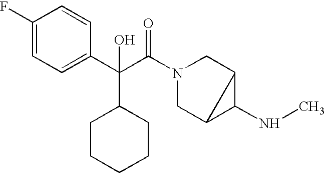 |
| 14. | 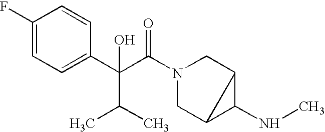 |
| 15. | 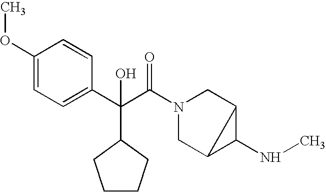 |
| 16. | 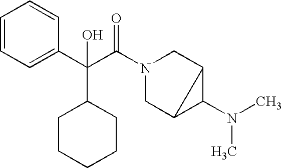 |
| 17. | 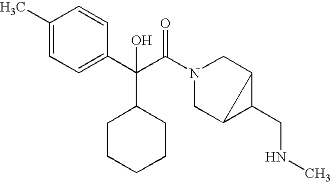 |
| 18. | 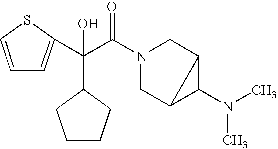 |
| 19. | 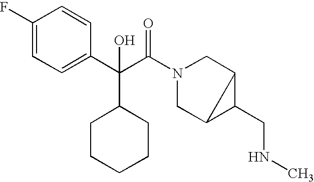 |
| 20. | 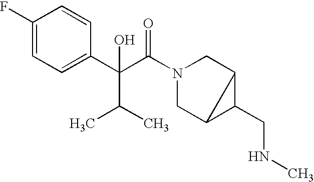 |
| 21. | 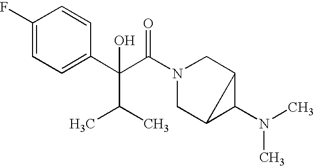 |
| 22. (1R) | 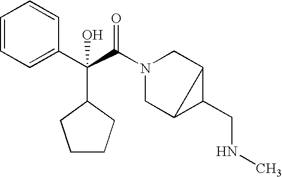 |
| 23. (1R) | 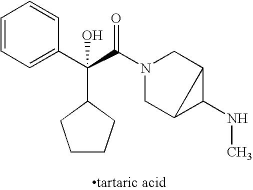 |
| 24. | 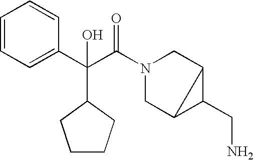 |
| 25. | 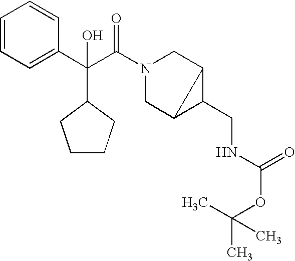 |
| 26. | 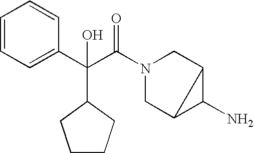 |
| 27. | 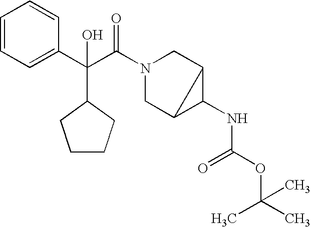 |
| 28. | 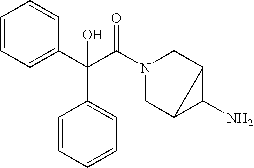 |
| 29. | 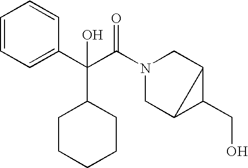 |
| 30. | 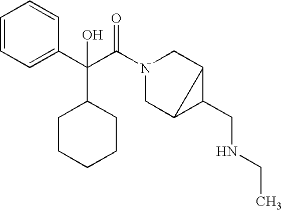 |
| 31. | 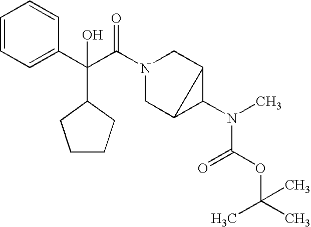 |
| 32. | 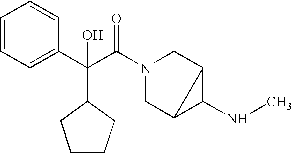 |
| 33. | 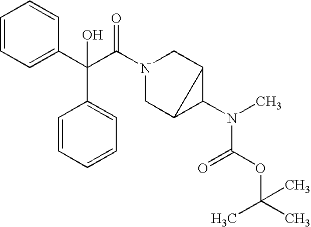 |
| 34. | 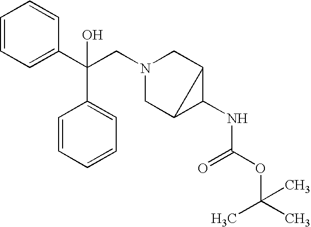 |
| 35. | 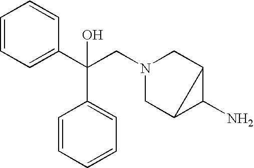 |
| 36. | 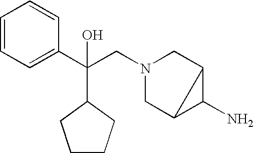 |
| 37. | 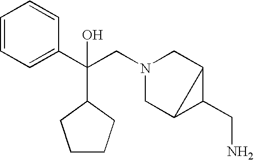 |
| 38. | 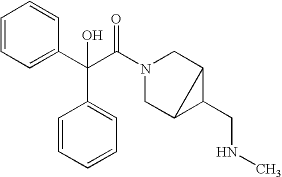 |
| 39. | 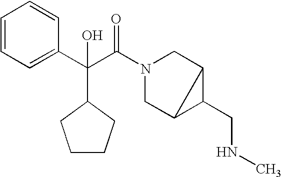 |
| 40. | 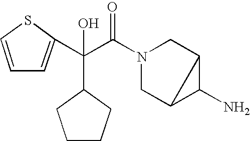 |
| 41. | 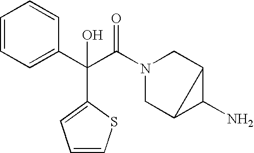 |
| 42. | 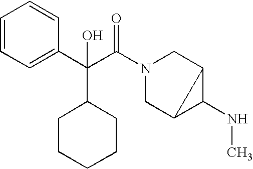 |
| 43. | 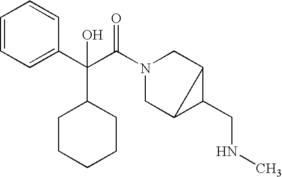 |
| 44. | 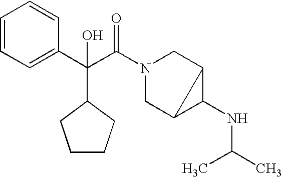 |
| 45. | 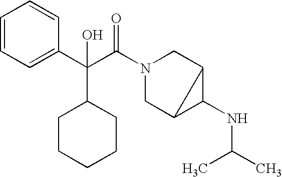 |
| 46. | 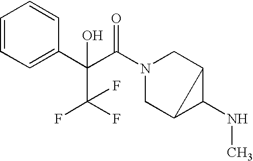 |
| 47. | 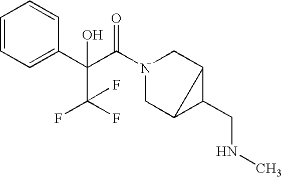 |
| 48. | 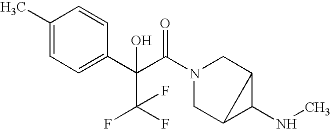 |
| 49. | 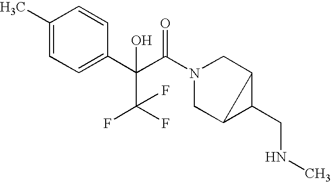 |
| 50. (1R or 1S) | 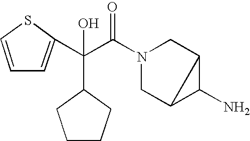 |

Because of their pharmacological properties, compounds described herein may be administered to an animal for treatment by any route, for example, orally or parenteral routes. The pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

Pharmaceutical compositions for use in the methods described herein may be prepared by any of the methods of pharmacy, but all methods include the step of bringing into association the active ingredient with the carrier which constitutes one or more necessary ingredients. In general, the compositions are prepared by uniformly and intimately admixing the active ingredient with pharmaceutically acceptable liquid carriers or finely divided solid carriers or both, and then, if necessary, shaping the product into the desired presentation.

Solid form preparations include powders, tablets, pills, dispersible granules, dragees, capsules, cachets, suppositories, troches, patches, gel caps, magmas, lozenges, creams, pastes, plasters, lotions, discs, or ointments. Liquid form preparations include solutions, suspensions, emulsions, microemulsions, syrups, elixirs, aerosols, nasal spays or oral sprays.

Solid carriers can include one or more substances, which may also act as diluents, flavoring agents, solubilizers, lubricants, suspending agents, binders, or disintegrating agents. Solid carriers can also include finely divided solids, which can be in admixture with one or more finely divided compounds described herein.

In preparing tablets, one or more compounds described herein can be mixed with one or more carriers having the necessary binding properties in suitable proportions and compacted into the desired shape and size. In some embodiments, powders and tablets can contain from about 5 to about 70 percent of one or more compounds described herein. Suitable solid carriers include, for example, sucrose, glucose, lactose, pectin, mannitol, silicic acid, dextrin, starch, gelatin, tragacanth, low melting wax, cocoa butter sugars, sodium citrate, dicalcium phosphate, microcrystalline cellulose, granulating agents, lubricants, binders, disintegrating agents, absorption accelerators, wetting agents, adsorbents and the like. Binders include, for example, carboxymethylcellulose, alginates, gelatins, polyvinylpyrrolidinone, sucrose, acacia; disintegrating agents include, for example, agar-agar, calcium carbonate, potato starch, alginic acid, certain silicates and sodium carbonate; absorption accelerators include, for example, quaternary ammonium compounds; wetting agents include, for example, acetyl alcohol, glycerol mono stearate; adsorbents include, for example, Kaolin; lubricants include, for example, talc, calcium stearate, magnesium stearate, solid polyethyleneglycol, sodium lauryl sulphate and mixture thereof. In the case of capsules, tablets, pills, the dosage form may also comprise buffering agents. For example, a tablet may be prepared by compression or molding, optionally, with one or more accessory ingredients. Compressed tablets may be prepared by compressing in a suitable machine the active ingredient in a free-flowing form such as powder or granules, optionally mixed with one or more binders, lubricants, inert diluents, surface active or dispersing agents. Molded tablets may be made by molding, in a suitable machine, a mixture of a powdered form of one or more compounds moistened with one or more inert liquid diluents.

For liquid form preparations, active compounds can be mixed with water or other solvent, solubilizing agents and emulsifiers, for example, ethyl alcohol, isopropanol, ethyl carbonate, ethyl acetate, benzyl alcohol, benzyl benzoate, propylene glycol, 1,3-butylene glycol, dimethylformamide, oils (for example, cottonseed, groundnut, corn, germ, olive, castor and sesame oil), glycerol, fatty acid esters of sorbitan or mixtures thereof.

Aqueous solutions suitable for oral use can be prepared by dissolving the active component in water and adding suitable colorants, flavors, stabilizers, wetting agents, emulsifying agents, suspending agents, sweetening agents, flavoring agents, perfuming agents and thickening agents as desired. Aqueous suspension suitable for oral use can be made by dispersing the finely divided active component in water with viscous material, for example, natural or synthetic gums, resins, methyl cellulose, sodium carboxymethyl cellulose and other suspending agents. Other liquid form preparations include, for example, water or water-propylene glycol solutions for parenteral injection. Other injectable preparations, for example, sterile injections, injectable depot forms, aqueous suspensions may be formulated according to the art using suitable dispersing or wetting and suspending agent. Among the acceptable vehicles and solvents that may be employed are water, Ringer's solution and isotonic sodium chloride.

Such solutions are prepared so as to be acceptable to biological systems with respect to isotonicity, pH, and other parameters. Liquid preparations can also be formulated in solution in aqueous polyethylene glycol solution.

Ointment preparations can contain one or more compounds described herein or salts thereof with a physiologically acceptable carrier. Such salts can be heavy metal salts. The carrier can desirably be a conventional water-dispersible hydrophilic or oil-in-water earner, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

Dosage forms for tropical or transdermal administration of one or more compounds described herein includes ointments, pastes, creams, lotions, gels, powders, solutions, sprays, inhalants or patches. Active compounds can be admixed under sterile condition with one or more pharmaceutically acceptable carriers and any desired preservatives or buffers as may be required. Ophthalmic formulations, eardrops, eye ointments, powders and solutions are also encompassed within the scope of this invention.

The pharmaceutical preparation can be in unit dosage form. In such forms, the preparation can be subdivided into unit doses containing appropriate quantities of the active component, i.e., one or more compounds described herein and optionally one or more other therapeutic agents. Dosage forms can be a packaged preparation containing one or more discrete unit dosages, for example, capsules; tablets; powders in vials, capsules or ampoules; ointments; cachets; gels or gel caps; cream itself; dispersible granules; suppositories; troches; patches; magmas; lozenges; pastes; plasters; lotions; discs; ointments; solutions; suspensions, emulsions, syrups, elixirs, aerosols, nasal spays or oral sprays.

The magnitude of a prophylactic or therapeutic dose of one or more compounds described herein in the acute or chronic prevention, treatment, or management of a disorder or condition will vary with the severity of the condition to be treated and the route of administration. The dose, and perhaps the dose frequency, will also vary according to the age, body weight, and response of the individual patient. Suitable total daily dose ranges can be readily determined by those skilled in the art. In general, the total daily dose range for one or more compounds described herein, for the conditions described herein, is from about 1 mg to about several grams administered in single or divided doses according to the particular application and the potency of the active ingredient. Compounds described herein can also be administered at initial dosages of about 3 mg to about 40 mg per kilogram daily. Suitable dosage amounts can be determined using small dosages that are less than the optimum dose. Such small dosages can be increased in small increments until the optimum effect is reached. Dosage amounts may be divided and administered as divided doses if desired.

Any suitable route of administration may be employed for providing the patient with an effective dosage of one or more compounds described herein according to the methods of the present invention. For example, oral, intraoral, rectal, parenteral, epicutaneous, transdermal, subcutaneous, intramuscular, intranasal, sublingual, buccal, intradural, intraocular, intrarespiratory, or nasal inhalation and like forms of administration may be employed. Oral administration is generally preferred.

In addition to the common dosage forms set out above, the compound for use in the methods of the present invention may also be administered by controlled release means and/or delivery devices such as those described in U.S. Pat. Nos. 3,845,770; 3,916,899; 3,536,809; 3,598,123; and 4,008,719, the disclosures of which are incorporated herein by reference.

The compounds described herein can be produced and formulated as their enantiomers, diastereomers, N-oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

Solvents used herein, such as acetone, methanol, pyridine, ether, tetrahydrofuran, hexanes and dichloromethane, were dried using various drying reagents according to procedures known to one of ordinary skill in the art. Infrared (1R) spectra were recorded as nujol mulls or a thin neat film on a Perkin Elmer Paragon instrument, Nuclear Magnetic Resonance (NMR) were recorded on a Varian XL-300 MHz instrument using tetramethylsilane as an internal standard.

### Example 1

### General Procedure: Synthesis of tert-butyl-3-azabicyclo[3.1.0]hex-6-yl(benzyl)carbamate

### Step a: Tert-butyl-(3-benzyl-3-azabicyclo[3.1.0]hex-6-yl)carbamate

Triethylamine (15.6 mL) and tert-butoxy carbonyl anhydride (17.1 mL) at room temperature was added to a solution of a compound (1α,5α,6α)-6-amino-3-benzyl-3-azabicyclo[3.1.0]hexane (prepared following the procedure as described in K. E. Brighty, Synlett, 1097-1102 at 1101 (1996)) (14 g) in dichloromethane (200 mL). The reaction mixture was stirred overnight followed by dilution with water. The aqueous layer was extracted with dichloromethane and the organic layer was washed with water followed by brine and dried over anhydrous sodium sulphate. The organic layer was evaporated under reduced pressure. The residue thus obtained was purified by column chromatography using 15% ethyl acetate in hexane as solvent mixture to yield the title compound. Yield=17 g.

### Step b: Tert-butyl-3-azabicyclo[3.1.0]hex-6-yl carbamate

A catalytic amount (0.05 g) of palladium on carbon (10%) was added to a solution of a compound obtained from step a above (10 g) in methanol (150 mL) and the resulting reaction mixture was stirred overnight under a hydrogen atmosphere. The reaction mixture was filtered through a celite pad and washed with methanol. The solvent was evaporated under reduce pressure to yield the title compound. Yield= 6.7 g.

### Step c: Benzyl-6-[(tert-butoxy carbonyl)amino-3-azabicyclo[3.1.0]hexane-3-carboxylate

Benzyl chloroformate (18.3 mL) and triethylamine (9 mL) were added to a solution of the compound obtained from step b above (11.5 g) in dichloromethane (200 mL) at 0° C. The reaction mixture was stirred overnight at room temperature and quenched with water and dichloromethane. The organic layer was washed with water followed by brine, dried over anhydrous sodium sulphate and concentrated under reduced pressure. The residue thus obtained was purified by column chromatography using 35% ethylacetate in hexane solvent mixture to yield the title compound. Yield= 6.4 g.

### Step d: Benzyl-6-[benzyl(tert-butoxycarbonyl)amino]-3-azabicyclo[3.1.0] hexane-3-carboxylate

Sodium hydride (288 mg) and benzyl bromide (0.85 mL) was added to a solution of the compound obtained from step c above (1.2 g) in tetrahydrofuran (10 mL) under nitrogen atmosphere. The reaction mixture was stirred for 3 hours and quenched with aqueous ammonium chloride solution. The aqueous layer was extracted with ethylacetate and the organic layer was washed with water, dried over anhydrous sodium sulphate and concentrated under reduced pressure. The residue thus obtained was purified by column chromatography by using 15% ethylacetate in hexane solvent mixture to yield the title compound. Yield=1.3 g.

### Step e: Tert-butyl-3-azabicyclo[3.1.0]hex-6-yl(benzyl)carbamate

A mixture of potassium hydroxide and methanol (40%, 25 mL) and 3-4 drops of water was added to the compound obtained from step d above (1.6 g). The reaction mixture was heated at 100° C. for 4 hours and stirred overnight. The solvent was evaporated under reduced pressure and the residue thus obtained was purified by acid base extraction to yield the title compound

Analogues of tert-butyl-3-azabicyclo[3.1.0]hex-6-yl(benzyl)carbamate described below were prepared by reacting alkyl halide in place of benzyl bromide with an amine respectively, as applicable in each case.

### Tert-butyl-3-azabicyclo[3.1.0]hex-6-yl(methyl)carbamate

¹H NMR (CDCl3): 3.07-3.11 (d, 2H, J=11.4 Hz), 2.91-2.95 (d, 2H, J= 11.4 Hz), 2.81 (s, 3H), 2.21 (s, 1H), 1.67 (s, 2H), 1.46 (s, 9H).

### Tert-butyl-3-azabicyclo[3.1.0]hex-6-yl(allyl)carbamate

¹H NMR (CDCl3): 5.7 (m, 1H), 5.08 (m, 2H), 3.79 (m, 2H), 3.08 (m, 2H), 2.93 (m, 2H), 2.2 (s, 1H), 1.71 (s, 2H), 1.45 (s, 9H)

m/z: 239.2 (M+1).

### Example 2

### Synthesis of 3,3,3-trifluoro-2-hydroxy-2-(4-methylphenyl)propanoic acid

### Step a: Ethyl-3,3,3-trifluoro-2-hydroxy-2-(4-methylphenyl)propanoate

A solution of toluene (0.540 g, 5.88 mmol), ethyl-3,3,3-trifluoro-2-oxopropanoate (1.0 g) in dichloroethane (5 mL) and triflic acid (0.052 mL) was stirred overnight at room temperature. The reaction mixture was poured in saturated sodium bicarbonate solution and extracted with ethyl acetate. The organic layer was washed with brine, dried over anhydrous sodium sulphate and concentrated under reduced pressure. The residue thus obtained was purified by column chromatography using 2% ethyl acetate in hexane solvent mixture as eluent to yield the title compound. Yield= 940 mg.

### Step b: 3,3,3-Trifluoro-2-hydroxy-2-(4-methylphenyl)propanoic acid

Potassium hydroxide (0.962 g, 17.17 mmol) was added to a solution of the compound obtained from step a above (0.9 g) in methanol (30 mL) and the reaction mixture was stirred at room temperature overnight. The reaction mixture was concentrated under reduced pressure and the residue thus obtained was diluted with water and extracted with ether. The aqueous layer was acidified with hydrochloric acid and extracted with ethyl acetate. The organic layer was washed with water, dried over anhydrous sodium sulphate and concentrated under reduced pressure to yield the title compound. Yield= 0.55 g.

The analogue of 3,3,3-trifluoro-2-hydroxy-2-(4-methylphenyl)propanoic acid described below was prepared by using appropriate ester in place of ethyl-3,3,3-trifluoro-2-oxopropanoate respectively, as applicable in each case.

### 3,3,3-Trifluoro-2-hydroxy-2-phenylpropanoic acid

1HNMR (CDCl3)= 7.8 (m, 2H, Ar—H), 7.41-7.42 (m, 3H, Ar—H)

IR in KBr=1733.4 cm-1

### Example 3

### Synthesis of tert-butyl {3-[cyclopentyl(hydroxy)phenylacetyl]-3-azabicyclo [3.1.0]hex-6-yl}methylcarbamate (Compound No. 31)

A solution of a compound 2-cyclopentyl-2-hydroxy-phenylacetic acid (synthesis as per procedure reported in J. Am. Chem. Soc., 75:265 (1953) and EP 613232, (1.415 mmol, 1 eq.) and tert-butyl-3-azabicyclo[3.1.0]hex-6-yl(methyl)carbamate (1.415 mmol) in dimethylformamide (10 mL) was cooled in an ice bath followed by the addition of N-methylmorpholine (0.28 g) and 1-hydroxy benzotriazole (0.21 g). The reaction mixture was stirred for 1 hour in an ice bath, 1-(3-dimethylamino propyl)-3-ethyl carbodiimide hydrochloride (0.27 g) was added and the reaction mixture was stirred for 1 hour and subsequently overnight. The reaction mixture was poured in saturated sodium bicarbonate solution and extracted with ethyl acetate. The organic layer was washed with brine, dried over anhydrous sodium sulphate and concentrated under reduced pressure. The residue thus obtained was purified by column chromatography using 20% ethyl acetate in hexane solvent mixture to yield the title compound. Yield= 280 mg.

m.p: 76-79.6° C.

¹H NMR: δ 7.40-7.26 (m, 5H), 3.98-2.66 (m, 7H), 1.65-1.21 (m, 20H).

IR: 1623, 1700 cm⁻¹.

Analogues of tert-butyl {3-[cyclopentyl(hydroxy)phenylacetyl]-3-azabicyclo [3.1.0]hex-6-yl}methylcarbamate (Compound No. 31) described below can be prepared by condensing appropriate amine with an acid, respectively, as applicable in each case.

### a) Tert-butyl ({3-[cyclopentyl(hydroxy)phenylacetyl]-3-azabicyclo[3.1.0]hex-6-yl}methyl) carbamate (Compound No. 25)

¹H NMR: 7.33 (m, 5H), 5.26 (bs, 0.5H), 5.04 (bs, 0.5H), 4.2 (m, 1H), 3.87 (m, 1H), 3.5 (m, 2H), 3.2 (m, 1H), 2.92 (m, 3H), 2.55 (m, 1H), 1.7-1.24 (m, 17H).

IR (KBr): 3361.7, 1696.6 and 1621.8 cm⁻¹.

Mass (m/z): 415.5 (M⁺+1).

### b) Tert-butyl {3-[cyclopentyl(hydroxy)phenylacetyl]-3-azabicyclo[3.1.0]hex-6-yl}carbamate (Compound No. 27)

¹H NMR: 7.35 (m, 5H), 5.13 (m, 1H); 4.6 (m, 1H), 3.88 (m, 1H), 3.50 (m, 3H), 2.94 (m, 1H), 2.05 (m, 1H), 1.79-1.4 (m, 19H).

IR: 3336 and 1700 cm⁻¹.

Mass (m/z): 401 (M⁺+1).

### Example 4

### Synthesis of 1-cyclopentyl-2-[6-(dimethylamino)-3-azabicyclo[3.1.0]hex-3-yl]-2-oxo-1-phenylethanol (Compound No. 10)

### Step a: 3-Benzyl-N,N-dimethyl-3-azabicyclo[3.1.0]hexan-6-amine

3-benzyl-3-azabicyclo[3.1.0]hexan-6-amine (1.5 g) was cooled in an ice bath and formic acid (0.9 mL) was added followed by slow addition of formaldehyde solution (1.66 mL). The reaction mixture was stirred at 80° C. for 24 hours, cooled and acidified with 1N hydrochloric acid and then extracted with diethyl ether. The aqueous layer was separated and basified with aqueous sodium hydroxide and extracted with ether. The ether layer thus obtained was washed with water, dried over anhydrous sodium sulphate, filtered and concentrated under reduced pressure to yield the title compound. Yield= 1.47 g.

### Step b: N,N-dimethyl-3-azabicyclo[3.1.0]hexane-6-amine

A catalytic amount (0.05 g) of palladium or carbon (10%) was added to a solution of the compound obtained from step a above (1.47 g) in methanol (10 mL) and the reaction mixture was stirred under a hydrogen atmosphere at room temperature for 4 hours, filtered through a celite pad and washed with methanol. The filtrate was concentrate under reduced pressure to yield the title compound. Yield: 485 mg.

### Step c: 1-Cyclopentyl-2-[6-(dimethylamino)-3-azabicyclo[3.1.0]hex-3-yl]-2-oxo-1-phenylethanol

The title compound was prepared by following the procedure as described for the synthesis of Compound No. 31 by using the compound obtained from step b above in place of tert-butyl-3-azabicyclo[3.1.0]hex-6-yl(methyl)carbamate.

¹H NMR: δ 7.31 (m, 5H), 5.26-5.02 (m, 1H), 3.90-3.38 (m, 4H), 2.90 (m, 1H), 2.26-2.10 (m, 6H), 1.78-1.28 (m, 11H).

IR (DCM): 3408 & 1619 cm⁻¹.

Mass (m/z): 329.3 (M⁺+1).

Analogues of 1-cyclopentyl-2-[6-(dimethylamino)-3-azabicyclo[3.1.0]hex-3-yl]-2-oxo-1-phenylethanol (Compound No. 10) described below can be prepared by coupling appropriate with acid an amine, respectively, as applicable in each case.

### a) 1-Cyclohexyl-2-[6-(dimethylamino)-3-azabicyclo[3.1.0]hex-3-yl]-2-oxo-1-phenylethanol (Compound No. 16)

¹H NMR: δ 7.34 (m, 5H), 5.5-4.7 (m, 1H), 3.81-3.2 (m, 4H), 2.35-2.0 (m, 6H), 2.0-1.11 (m, 13H).

Mass (m/z): 343.3 (M⁺+1).

IR (DCM): 3396 and 1618 cm⁻¹.

m.p.: 71.3-72.7° C.

### b) 1-Cyclopentyl-2-[6-(dimethylamino)-3-azabicyclo[3.1.0]hex-3-yl]-2-oxo-1-(2-thienyl)ethanol (Compound No. 18)

1H NMR: δ 7.26 (m, 1H), 6.95 (m, 2H), 5.5 (m, 1H), 3.91-3.35 (m, 3H), 2.82 (m, 1H), 2.2 (m, 6H), 1.89-1.28 (m, 1H), 0.88 (m, 1H).

### c) 2-(4-fluorophenyl)-1-[6-(dimethylamino)-3-azabicyclo[3.1.0]hex-3-yl]-3-methyl-1-oxobutan-2-ol (Compound No. 21)

¹H NMR: δ 7.38 (m, 2H), 7.04 (m, 2H), 3.9-3.2 (m, 5H), 2.73 (m, 2H), 2.2 (m, 6H), 1.5 (m, 2H), 0.9 (m, 5H).

Mass (m/z): 321 (M⁺+1).

### Example 5

### Synthesis of 2-[6-(benzylamino)-3-azabicyclo[3.1.0]hex-3-yl]-1-cyclopentyl-2-oxo-1-phenylethanol (Compound No. 1)

### Step a: Tert-butyl-benzyl-{3-[cyclopentyl(hydroxy)phenylacetyl]-3-azabicyclo [3.1.0]hex-6-yl}-carbamate

The title compound was prepared following the procedure as described for the synthesis of Compound No. 31 by using tert-butyl-3-azabicyclo[3.1.0]hex-6-yl(benzyl)carbamate in place of tert-butyl-3-azabicyclo[3.1.0]hex-6-yl(methyl)carbamate.

### Step b: 2-[6-(Benzylamino)-3-azabicyclo[3.1.0]hex-3-yl]-1-cyclopentyl-2-oxo-1-phenylethanol

A methanolic solution of hydrochloric acid (10 mL) was added to the compound obtained from step a above (140 mg) and the reaction mixture was stirred for 5 hours. The reaction mixture was concentrated under reduced pressure and the residue thus obtained was diluted with water. The aqueous layer was basified with 10% aqueous sodium hydroxide and extracted with ethyl acetate. The organic layer was separated, washed with water and brine, dried over anhydrous sodium sulphate and concentrated under reduced pressure to yield the title compound. Yield=92 mg.

¹H NMR: δ 7.33-7.1 (m, 10H), 3.82-3.39 (m, 5H), 3.21 (m, 1H), 2.87 (m, 1H) and 1.8-1.28 (m, 12H).

IR (DCM): 3421.8, 1621.7 cm⁻¹.

Mass (m/z): 391.48 (M⁺+1).

Analogues of 2-[6-(benzylamino)-3-azabicyclo[3.1.0]hex-3-yl]-1-cyclopentyl-2-oxo-1-phenylethanol (Compound No. 1) described below can be prepared by deprotecting appropriate amine respectively, as applicable in each case.

### a) 2-[6-(Benzylamino)-3-azabicyclo[3.1.0]hex-3-yl]-1-cyclohexyl-2-oxo-1-phenylethanol (Compound No. 2)

¹H NMR: δ 7.37-7.13 (m, 10H), 4.69 (s, 1H), 3.80-3.37 (m, 6H), 2.33 (m, 1H), 1.92-1.68 (m, 7H), 1.43-1.08 (m, 4H).

IR: 3384, 1618.3 cm⁻¹.

Mass (m/z): 405.3 (M⁺+1).

### b) 2-(6-Amino-3-azabicyclo[3.1.0]hex-3-yl)-1-cyclopentyl-1-(4-methylphenyl)-2-oxoethanol (Compound No. 3)

¹H NMR: δ 7.25 (d, J=7.8 Hz, 2H), 7.12 (d, J=7.8 Hz, 2H), 3.83 (m, 1H), 3.45 (m, 2H), 3.20 (m, 1H), 2.88 (m, 1H), 2.60 (m, 1H), 2.33 (m, 4H), 1.89-1.23 (m, 9H).

Mass (m/z): 315.3 (M⁺+1).

### c) 2-[6-(Allylamino)-3-azabicyclo[3.1.0]hex-3-yl]-1-cyclopentyl-1-(4-methylphenyl)-2-oxoethanol (Compound No. 6)

¹H NMR: δ 7.25 and 7.12 (d, J=6 Hz, 2H each), 5.75 (m, 1H), 5.02 (m, 2H), 3.75 (m, 1H), 3.42 (m, 2H), 3.21 (m, 1H), 3.07 (m, 1H), 2.89 (m, 1H), 2.33 (s, 3H), 1.8-1.28 (m, 11H).

### d) 2-[6-(Allylamino)-3-azabicyclo[3.1.0]hex-3-yl]-1-cyclopentyl-2-oxo-1-phenylethanol (Compound No. 7)

¹H NMR: δ 7.35 (m, 5H), 5.75 (m, 1H), 5.0 (m, 2H), 3.85 (m, 1H), 3.50-2.92 (m, 5H), 1.82-1.28 (m, 12H).

IR: 3384.7 and 1619 cm⁻¹.

Mass (m/z): 341.3 (M⁺+1).

### e) 1-Cyclohexyl-2-[6-(methylamino)-3-azabicyclo[3.1.0]hex-3-yl]-1-(4-methylphenyl)-2-oxoethanol (Compound No. 12)

¹H NMR: δ 7.27 & 7.13 (d, J=8.1 Hz, 2H each), 3.79-3.21 (m, 3H), 2.9 (m, 1H), 2.32 (m, 7H), 1.82-1.6 (m, 5H), 1.44-0.88 (m, 7H).

IR (KBr): 3406, 1618 cm⁻¹.

Mass (m/z): 343.2 (M⁺+1).

### f) 1-Cyclohexyl-1-(4-fluorophenyl)-2-[6-(methylamino)-3-azabicyclo[3.1.0]hex-3-yl]-2-oxoethanol (Compound No. 13)

¹H NMR: δ 7.37 (m, 2H), 7.03 (m, 2H), 3.78 (m, 1H), 3.5-3.2 (m, 3H), 2.29 (m, 4H), 1.78 (m, 5H), 1.47-1.26 (m, 8H).

IR (KBr): 3426.3 and 1619.2 cm⁻¹.

Mass (m/z): 347 (M⁺+1).

### g) 2-(4-Fluorophenyl)-3-methyl-1-[6-(methylamino)-3-azabicyclo[3.1.0]hex-3-yl]-1-oxobutan-2-ol (Compound No. 14)

¹H NMR: δ 7.40 (m, 2H), 7.03 (m, 2H), 3.79 (m, 1H), 3.48-3.25 (m, 3H), 2.73 (m, 2H), 2.32 (m, 3H), 1.47 (m, 2H), 0.9 (m, 6H).

### h) 1-Cyclopentyl-1-(4-methoxyphenyl)-2-[6-(methylamino)-3-azabicyclo[3.1.0]hex-3-yl]-2-oxoethanol (Compound No. 15)

¹H NMR: δ 7.28 (m, 2H), 6.85 (m, 2H), 3.80 (m, 4H), 3.46-3.24 (m, 3H), 2.89 (m, 1H), 2.40-2.26 (m, 4H), 1.76-1.25 (m, 10H).

IR (DCM): 3376.6 and 1613.9 cm⁻¹.

Mass (m/z): 345.2 (M⁺+1).

### i) 1-Cyclohexyl-2-{6-[(methylamino)methyl]-3-azabicyclo[3.1.0]hex-3-yl}-1-(4-methylphenyl)-2-oxoethanol (Compound No. 17)

¹H NMR: δ 7.26 (m, 2H), 7.11 (m, 2H), 3.82-3.2 (m, 4H), 2.32 (m, 8H), 2.0 (m, 2H), 1.76 (m, 4H), 1.28 (m, 8H).

IR (DCM): 3406.2 and 1618.1 cm⁻¹.

Mass (m/z): 357.2 (M+1).

### j) 1-Cyclohexyl-1-(4-fluorophenyl)-2-{6-[(methylamino)methyl]-3-azabicyclo[3.1.0]hex-3-yl}-2-oxoethanol (Compound No. 19)

¹H NMR: δ 7.37 and 7.01 (m, 2H each), 3.9-3.2 (m, 4H), 2.2 (m, 7H), 1.76 (m, 4H), 1.31 (m, 8H).

Mass (m/z): 361.2 (MM).

### k) 2-(4-Fluorophenyl)-3-methyl-1-{6-[(methylamino)methyl]-3-azabicyclo[3.1.0]hex-3-yl}-1-oxobutan-2-ol (Compound No. 20)

¹H NMR: δ 7.38 (m, 2H), 7.02 (m, 2H), 3.9-3.2 (m, 4H), 2.72 (m, 2H), 2.38 (m, 5H), 1.28 (m, 2H), 0.92 (m, 6H).

IR (DCM): 3384.4, 1616.8 cm⁻¹.

Mass (m/z): 321.2 (M⁺+1).

### l) 2-[6-(Aminomethyl)-3-azabicyclo[3.1.0]hex-3-yl]-1-cyclopentyl-2-oxo-1-phenylethanol (Compound No. 24)

¹H NMR: δ 7.36 (m, 5H), 3.88 (m, 1H), 3.4 (m, 2H), 2.9 (m, 1H), 2.45 (m, 2H), 1.79-1.1 (m, 12H).

Mass (m/z): 315 (M⁺+1).

### m) 2-(6-Amino-3-azabicyclo[3.1.0]hex-3-yl)-1-cyclopentyl-2-oxo-1-phenylethanol (Compound No. 26)

¹H NMR: δ 7.33 (m, 5H), 5.4 (bs, 0.5H), 5.0 (bs, 0.5H), 3.84 (m, 1H), 3.3 (m, 3H), 2.93 (m, 1H), 1.9-1.3 (m, 11H).

IR (DCM): 3362 and 1619 cm⁻¹.

Mass (m/z): 301.5 (M⁺+1).

### n) 2-(6-Amino-3-azabicyclo[3.1.0]hex-3-yl)-1-cyclohexyl-2-oxo-1,1-diphenylethanol (Compound No. 28)

¹H NMR: δ 7.30 (m, 10H), 3.9 (m, 1H), 3.54 (m, 1H), 3.17 (s, 1H), 2.99 (m, 1H), 2.54 (m, 1H), 1.93 (m, 1H), 1.5 (m, 2H).

IR: 3360 and 1623.7 cm⁻¹.

Mass (m/z): 309 (M+1).

### o) 1-Cyclopentyl-2-[6-(methylamino)-3-azabicyclo[3.1.0]hex-3-yl]-2-oxo-1-phenylethanol (Compound No. 32)

¹H NMR: δ 7.30-7.40 (m, 5H), 3.77-3.89 (m, 1H), 3.24-3.31 (m, 2H), 2.93 (bs, 1H), 2.24-2.38 (m, 3H), 1.71-1.20 (m, 12H).

IR (KBr): 1615 cm⁻¹.

### p) Tert-butyl {3-[hydroxy(diphenyl)acetyl]-3-azabicyclo[3.1.0]hex-6-yl}methylcarbamate (Compound No. 33)

¹H NMR: δ 7.38-7.33 (m, 10H), 5.62 (s, 1H), 4.07-4.12 (d, 1H), 3.65-3.70 (dd, 1H), 3.16-3.20 (d, 1H), 2.74 (s, 3H), 2.66-2.69 (d, 1H), 2.28 (m, 1H), 2.06 (s, 1H), 1.6 (m, 2H), 1.40 (s, 9H).

### q) 2-{6-[(Methylamino)methyl]-3-azabicyclo[3.1.0]hex-3-yl}-2-oxo-1,1-diphenylethanol (Compound No. 38)

¹H NMR: δ 7.34 (s, 5H), 5.09 (s, 2H), 3.68-3.60 (t, 2H), 3.43-3.39 (t, 2H), 3.05-3.01 (m, 2H), 1.5 (s, 2H), 1.44 (s, 9H), 1.25 (s, 1H).

### r) 1-Cyclopentyl-2-{6-[(methylamino)methyl]-3-azabicyclo[3.1.0]hex-3-yl}-2-oxo-1-phenyl ethanol (Compound No. 39)

¹H NMR: δ 7.19-7.30 (m, 5H), 4.89 (bs, 1H), 3.82 (m, 1H), 3.35-2.45 (m, 3H), 2.33-1.93 (m, 8H), 1.65-1.19 (m, 10H).

### s) 2-(6-Amino-3-azabicyclo[3.1.0]hex-3-yl)-1-cyclopentyl-2-oxo-1-(2-thienyl)ethanol (Compound No. 40)

¹H NMR: δ 7.01-6.92 (m, 3H), 3.79-2.87 (m, 7H), 2.17-1.005 (m, 10H).

### t) 2-(6-Amino-3-azabicyclo[3.1.0]hex-3-yl)-2-oxo-1-phenyl-1-(2-thienyl)ethanol (Compound No. 41)

¹H NMR: δ 7.38-7.34 (m, 6H), 7.01-6.44 (m, 2H), 4.038-2.54 (m, 5H), 1.25 (s, 2H).

### u) 1-Cyclohexyl-2-[6-(methylamino)-3-azabicyclo[3.1.0]hex-3-yl]-2-oxo-1-phenylethanol (Compound No. 42)

¹H NMR: δ 7.41-7.22 (m, 5H), 3.79-2.81 (m, 4H), 2.35-2.25 (m, 3H), 1.82-1.11 (m, 14H).

IR (DCM): 1616 cm⁻¹.

### v) 1-Cyclohexyl-2-{6-[(methylamino)methyl]-3-azabicyclo[3.1.0]hex-3-yl}-2-oxo-1-phenyl ethanol (Compound No. 43)

¹H NMR: δ 7.40-7.21 (m, 5H), 3.83-2.52 (m, 4H), 2.34 (m, 6H), 1.94-1.10 (m, 13H).

IR (DCM): 1617 cm⁻¹.

m.p: 140-142° C.

### x) 1-Cyclopentyl-2-[6-(isopropylamino)-3-azabicyclo[3.1.0]hex-3-yl]-2-oxo-1-phenyl ethanol (Compound No. 44)

¹H NMR: δ 7.40-7.26 (m, 5H), 3.94-2.66 (m, 6H), 1.83-1.03 (m, 17H).

IR (DCM): 1619 cm⁻¹.

### y) 1-Cyclohexyl-2-[6-(isopropylamino)-3-azabicyclo[3.1.0]hex-3-yl]-2-oxo-1-phenylethanol (Compound No. 45)

¹H NMR: δ 7.41-7.26 (m, 5H), 3.83-2.35 (m, 7H), 1.66-0.908 (m, 19H).

### z) 1,1,1-Trifluoro-3-[6-(methylamino)-3-azabicyclo[3.1.0]hex-3-yl]-3-oxo-2-phenylpropan-2-ol (Compound No. 46)

¹H NMR: δ 7.41 (s, 5H), 4.09-3.08 (m, 5H), 2.56 (m, 1H), 2.36-2.32 (d, J= 12 Hz, 3H), 2.08-2.02 (m, 2H).

m.p: 179.7-182° C.

### aa) 1,1,1-Trifluoro-3-{6-[(methylamino)methyl]-3-azabicyclo[3.1.0]hex-3-yl}-3-oxo-2-phenylpropan-2-ol (Compound No. 47)

¹H NMR: δ 7.46-7.39 (s, 5H), 4.01-3.97 (m, 1H), 3.62-3.23 (m, 3H), 2.76-2.36 (m, 8H), 1.29 (m, 2H).

m.p: 110-113° C.

### ab) 1,1,1-Trifluoro-3-[6-(methylamino)-3-azabicyclo[3.1.0]hex-3-yl]-2-(4-methylphenyl)-3-oxopropan-2-ol (Compound No. 48)

¹H NMR: δ 7.31-7.20 (m, 5H), 4.01-3.97 (m, 1H), 3.56 (m, 1H), 3.35-3.08 (m, 2H), 2.63-2.37 (m, 7H), 1.24 (m, 2H).

mp: 178-179.4° C.

### ac) 1,1,1-Trifluoro-3-{6-[(methylamino)methyl]-3-azabicyclo[3.1.0]hex-3-yl}-2-(4-methylphenyl)-3-oxopropan-2-ol (Compound No. 49)

¹H NMR: δ 7.34-7.18 (m, 5H), 3.98-3.95 (m, 1H), 3.57-3.33 (m, 2H), 2.82-2.36 (m, 10H), 1.25 (m, 2H).

m.p: 191-194° C.

### Example 6

### Synthesis of (1R or 1S)-2-(6-amino-3-azabicyclo[3.1.0]hex-3-yl)-1-cyclopentyl-2-oxo-1-(2-thienyl)ethanol (Compound No. 50)

### Step a: (−)-2-Cyclopentyl-2-hydroxy-2-(2-thienyl)acetic acid

A solution of 2-Cyclopentyl-2-hydroxy-2-(2-thienyl)acetic acid (4.2 g) and quinine (6.028 g) in ethanol (200 mL) was heated and then allowed to cool. The salt thus formed was crystallized three times from ethanol. The salt (1.5 g) dissolved in dilute hydrochloric acid and extracted with dichloromethane. The organic layer was washed with water, dried over anhydrous sodium sulphate and concentrated under reduced pressure to yield the title compound. Yield= 550 mg. [α]_{D}=14.5 (1%, MeOH).

### Step b: Tert-butyl{3-[cyclopentyl(hydroxy)-2-thienyl-acetyl]-3-azabicyclo[3.1.0]hex-6-yl}carbamate

The title compound was prepared following the procedure as described for the synthesis of compound No. 31 by using (−)-2-cyclopentyl-2-hydroxy-2-(2-thienyl)acetic acid in place of 2-cyclopentyl-2-hydroxy-phenyl acid and using tert-butyl-3-azabicyclo[3.1.0]hex-6-yl carbamate in place of tert-butyl-3-azabicyclo[3.1.0]hex-6-yl(methyl) carbamate.

### Step c: 2-(6-Amino-3-azabicyclo[3.1.0]hex-3-yl)-1-cyclopentyl-2-oxo-1-(2-thienyl)ethanol

The title compound was prepared following the procedure as described for the synthesis of Compound No. 1 (Example 2, Step b) by deprotecting a compound obtained from step b above. Yield: 60 mg.

¹H NMR: δ 7.02-6.93 (m, 3H), 3.49-3.41 (m, 5H), 2.85 (m, 2H), 1.72-1.11 (m, 10H).

### Example 7

### Synthesis of (1R)-1-cyclopentyl-2-{6-[(methylamino)methyl]-3-azabicyclo[3.1.0] hex-3-yl}-2-oxo-1-phenyl ethanol (Compound No. 22)

### Step a: Tert-butyl ({3-[(2R)-2-cyclopentyl-2-hydroxy-2-phenylacetyl]-3-azabicyclo[3.1.0]hex-6-yl}methyl carbamate

The title compound was prepared following the procedure as described for the synthesis of compound No. 31 by using (2R)-cyclopentyl (hydroxy) phenyl acetic acid in place of 2-cyclopentyl-2-hydroxy-phenyl acetic acid and using tert-butyl (3-azabicyclo[3.1.0]hex-6-ylmethyl)methyl carbamate in place of tert-butyl-3-azabicyclo[3.1.0]hex-6-yl (methyl) carbamate.

### Step b: 1-Cyclopentyl-2-{6-[(methylamino)methyl]-3-azabicyclo[3.1.0]hex-3-yl}-2-oxo-1-phenyl ethanol

The title compound was prepared following the procedure as described for the synthesis of compound No. 1 (Example 2, step b) by deprotecting compound obtained from step a above.

¹H NMR: δ 7.29 (m, 5H), 3.9-3.41 (m, 4H), 2.91 (m, 1H), 2.6-2.0 (m, 7H), 1.78-1.26 (m, 11H).

IR (DCM): 3356 and 1619.5 cm⁻¹.

Mass (m/z): 329.2 (M⁺+1).

### Example 8

### Synthesis of Tartarate Salt of (1R)-1-cyclopentyl-2-[6-(methylamino)-3-azabicyclo[3.1.0]hex-3-yl]-2-oxo-1-phenylethanol (Compound No. 23)

### Step a: Tert-butyl {3-[(2R)-2-cyclopentyl-2-hydroxy-2-phenylacetyl]-3-azabicyclo[3.1.0]hex-6-yl}methyl carbamate

The title compound was prepared following the procedure as described for the synthesis of Compound No. 31 by using (2R)-cyclopentyl (hydroxy) phenyl acetic acid in place of 2-cyclopentyl-2-hydroxy phenyl acetic acid.

### Step b: (1R)-1-cyclopentyl-2-[6-(methylamino)-3-azabicyclo[3.1.0]hex-3-yl]-2-oxo-1-phenylethanol

The title compound was prepared following the procedure as described for the synthesis of Compound No. 1 (Example 2, step b) by deprotecting the compound obtained from step b above.

### Step c: Tartarate salt of 1-cyclopentyl-2-[6-(methylamino)-3-azabicyclo[3.1.0]hex-3-yl]-2-oxo-1-phenylethanol

A solution of tartaric acid (1 mmol) in ethanol (5 mL) was added to a solution of the compound obtained from step b above (1 mmol) in ethanol (10 mL). The reaction mixture was stirred at 60° C. for 1 hour and concentrated under reduced pressure. The resulting solid was triturated with diethyl ether and diethyl ether was removed by decanting. The residual ether was removed under reduced pressure to yield the title compound. Yield: 339 mg.

¹H NMR: δ 7.29 (m, 5H), 4.37 (s, 2H), 4.0-3.27 (m, 6H), 2.77-2.47 (m, 4H), 1.95 (m, 2H), 1.80 (m, 1H), 1.54-1.2 (m, 6H).

Analogs of tartarate salt of (1R)-1-cyclopentyl-2-[6-(methylamino)-3-azabicyclo[3.1.0]hex-3-yl]-2-oxo-1-phenylethanol (Compound No. 23) described below can be prepared by using appropriate amine respectively, as applicable in each case.

### a) Tartarate Salt of 2-[6-(allylamino)-3-azabicyclo[3.1.0]hex-3-yl]-1-cyclohexyl-2-oxo-1-phenylethanol (Compound No. 4)

¹H NMR: δ 7.29 (m, 5H), 5.8-5.6 (m, 1H), 5.4-5.2 (m, 2H), 4.36 (s, 2H), 3.7 (m, 1H), 3.57 (m, 1H), 3.34-3.25 (m, 4H), 2.18 (m, 1H), 1.86-1.69 (m, 4H), 1.56 (m, 2H), 1.34-0.88 (m, 7H).

### b) Tartarate salt of 2-[6-(allylamino)-3-azabicyclo[3.1.0]hex-3-yl]-1-cyclohexyl-1-(4-methylphenyl)-2-oxoethanol (Compound No. 5)

¹H NMR: δ 7.21-7.09 (m, 4H), 5.75-5.5 (m, 1H), 5.25 (m, 2H), 4.35 (s, 2H), 3.57-3.25 (m, 6H), 2.27 (s, 3H), 2.16 (m, 1H), 1.87-1.5 (m, 6H), 1.45-0.9 (m, 7H).

### c) Tartarate Salt of 2-[6-(allylamino)-3-azabicyclo[3.1.0]hex-3-yl]-1-cyclopentyl-1-(4-fluorophenyl)-2-oxoethanol (Compound No. 8)

¹H NMR: δ 7.36 (m, 2H), 7.03 (m, 2H), 5.75 (m, 1H), 5.25 (m, 2H), 4.37 (s, 2H), 3.9-3.3 (m, 3H), 2.73 (m, 1H), 1.87 (m, 3H), 1.48-1.1 (m, 9H).

d) Tartarate salt of 1-[6-(allylamino)-3-azabicyclo[3.1.0]hex-3-yl]-3-methyl-1-oxo-2-phenylbutan-2-ol (Compound No. 9)

¹H NMR: δ 7.21 (m, 5H), 5.8-5.6 (m, 1H), 5.35-5.16 (m, 2H), 4.32 (s, 2H), 3.94-3.2 (m, 6H), 2.50 (m, 1H), 1.87 (m, 2H), 0.9 (m, 3H), 0.45 (m, 3H).

### Example 9

### Synthesis of 1-cyclobutyl-2-oxo-1-phenyl-2-[6-(propylamino)-3-azabicyclo [3.1.0]hex-3-yl]ethanol (Compound No. 11)

### Step a: Tert-butyl allyl {3-[cyclobutyl(hydroxy)phenylacetyl]-3-aza-bicyclo[3.1.0]hex-6-yl}carbamate

The title compound was prepared following the procedure as described for the described for the synthesis of Compound No. 31 by condensing cyclobutyl (hydroxy) phenyl acetic acid with tert-butyl allyl (3-azabicyclo[3.1.0]hex-6-yl)carbamate.

### Step b: Tert-butyl {3-[cyclobutyl(hydroxy)phenyl acetyl]-3-azabicyclo[3.1.0]hex-6-yl}propyl carbamate

A catalytic amount (0.05 g) of palladium on carbon (10%) was added to a solution of the compound obtained from step a above (750 mg) in methanol (15 mL) and the resulting reaction mixture was stirred under a hydrogen atmosphere overnight, filtered through a celite pad and washed with methanol. The filtrate was evaporated under reduced pressure and the residue thus obtained was purified by column chromatography by using 15% ethylacetate in hexane solvent mixture to yield the title compound. Yield: 468 mg.

### Step c: 1-Cyclobutyl-2-oxo-1-phenyl-2-[6-(propylamino)-3-azabicyclo[3.1.0]hex-3-yl]ethanol

The title compound was prepared by following the procedure as described for the synthesis of Compound No. 1 (Example 1, path b) by deprotecting the compound obtained from step b above. Yield: 305 mg.

¹H NMR: δ 7.30 (m, 5H), 3.9-3.8 (m, 1H), 3.43-3.13 (m, 4H), 2.57-2.4 (m, 4H), 2.04-1.74 (m, 5H), 1.45-1.37 (m, 4H), 0.89-0.81 (m, 3H).

### Example 10

### Synthesis of 1-cyclohexyl-2-[6-(hydroxymethyl)-3-azabicyclo[3.1.0]hex-3-yl]-2-oxo-1-phenylethanol (Compound No. 29)

The title compound was prepared following the procedure as described for the synthesis of Compound No. 31 by condensing cyclohexyl (hydroxy) phenyl acetic acid with 3-azabicyclo[3.1.0]hex-6-ylmethanol.

¹H NMR (CDCl₃): 7.30 (m, 5H), 5.1 (bs, 0.5H), 4.75 (bs, 0.5H), 3.7-3.3 (m, 6H), 2.36 (m, 1H), 1.77 (m, 4H), 1.3 (m, 9H).

IR (DCM): 3314, 1594.8 cm⁻¹.

Mass (m/z): 330 (M⁺+1)

### Example 11

### Synthesis of 1-cyclohexyl-2-{6-[(ethylamino)methyl]-3-azabicyclo[3.1.0]hex-3-yl}-2-oxo-1-phenylethanol (Compound No. 30)

### Step a: {3-(2-Cyclohexyl-2-hydroxy-2-phenylacetyl)-3-azabicyclo-[3.1.0]hex-6-yl]methyl methane sulphonate

A catalytic amount (0.05 g) of diethylaminopyridine and triethylamine (368 mg) was added to a solution of Compound No. 29 (600 mg) in dry dichloromethane (15 mL). The resulting reaction mixture was cooled to 0° C. to −5° C. and methane sulphonyl chloride (211 μL) was added slowly. The reaction mixture was stirred at 0° C. for 2 hours and then stirred at room temperature overnight. The reaction mixture was diluted with dichloromethane and quenched with saturated solution of sodium bicarbonate. The organic layer was separated, washed with water, brine, dried over anhydrous sodium sulphate, filtered and evaporated the organic layer under reduced pressure to yield the title compound. Yield: 675 mg.

### Step b: 1-Cyclohexyl-2-{6-[(ethylamino)methyl]-3-azabicyclo[3.1.0]hex-3-yl}-2-oxo-1-phenylethanol

Ethylamine (70% aqueous solution, 2 mL) was added to a solution of the compound obtained from step a above (650 mg) in ethanol (5 mL) and the reaction mixture was stirred in a tightened steel bomb/reaction vessel at 80° C. for 8-9 hours. The reaction mixture was cooled to −78° C. and steel bomb was opened up. The organic solvent was evaporated under reduced pressure and diluted with dilute hydrochloric to a pH of 1-2. The aqueous layer was extracted with ethylacetate and the aqueous layer was basified with 10% aqueous sodium hydroxide solution to a pH of 12-13. The basified aqueous layer was extracted with dichloromethane. The organic layer was dried over anhydrous sodium sulphate, filtered and evaporated under reduced pressure to yield the title compound. Yield: 228 mg.

¹H NMR (CDCl₃): 7.4-7.21 (m, 5H), 3.83-3.64 (m, 2H), 3.36-2.7 (m, 6H), 2.61 (m, 4H), 2.34 (m, 1H), 1.74 (m, 4H), 1.4-1.18 (m, 11H).

IR (KBr): 3423.9 and 1616.2 cm⁻¹

Mass (m/z): 357.5 (M⁺+1).

m.p.: 111.8-114.8° C.

### Example 12

### Synthesis of tert-butyl [3-(2-hydroxy-2,2-diphenylethyl)-3-azabicyclo[3.1.0]hex-6-yl]carbamate (Compound No. 34)

### Step a: 2,2-Diphenyl oxirane

Sodium hydride (1.42 g, 35.62 mmol) was added to a solution of the compound benzophenone (5 g, 27.4 mL) and trimethyl sulphoxonium iodide (6.95 g, 30.2 mmol) in dimethyl sulphoxide (30 mL) at room temperature and the reaction mixture was stirred for 30 minutes at room temperature followed by stirring at 50° C. for 6 hours and then stirring overnight at room temperature. The resulting mixture was poured into water and extracted with ethyl acetate. The organic layer was concentrated under reduced pressure to yield the title compound. Yield: 4.7 g.

### Step b: Tert-butyl [3-(2-hydroxy-2,2-diphenylethyl)-3-azabicyclo[3.1.0]hex-6-yl]carbamate

Potassium carbonate (1.41 g) was added to a solution of tert-butyl-3-azabicyclo[3.1.0]hex-6-ylcarbamate (0.84 g, 4.25 mmol) in dimethylformamide (15 mL) and the reaction mixture was stirred for 45 minutes under nitrogen atmosphere. A compound obtained from step a above (1 g, 5.102 mmol) was added to the resulting reaction mixture and stirred for 9 hours at 90-100° C. followed by stirring at room temperature overnight. The reaction mixture was poured into water and extracted with ethyl acetate. The organic layer was washed with water followed by brine, dried over anhydrous sodium sulphate and concentrated under reduced pressure. The residue thus obtained was purified by column chromatography using 20% ethyl acetate in hexane solvent mixture to yield the title compound. Yield: 270 mg.

¹H NMR: δ 7.15-7.49 (m, 10H), 3.3 (s, 2H), 2.78-2.84 (m, 2H), 2.67 (bs, 1H), 2.43-2.46 (m, 2H), 1.59-1.62 (m, 2H), 1.41 (s, 9H).

m.p: 127-131° C.

### Example 13

### Synthesis of 2-(6-amino-3-azabicyclo[3.1.0]hex-3-yl)-1,1-diphenylethanol (Compound No. 35)

A solution of the Compound No. 34 (0.235 g) in ethyl acetate (10 mL) was cooled in an ice bath followed by the addition of solution of hydrochloric acid in ethyl acetate (10 mL) and the resulting solution became turbid. The reaction mixture was stirred at room temperature for 3 hours. A solution of hydrochloric acid in ethylacetate (10 mL) and few drops of methanol were added to the resulting reaction mixture. The reaction mixture was stirred at room temperature overnight, concentrated under reduced pressure and the residue thus obtained was dissolved in water and washed with dichloromethane. The aqueous layer was basified with sodium hydroxide solution (10%) and extracted with ethyl acetate. The organic layer was dried and concentrated under reduced pressure to yield the title compound. Yield: 150 mg.

¹H NMR: δ 7.49-7.25 (m, 4H), 7.30-7.15 (m, 7H), 3.29 (s, 2H), 2.69-2.66 (d, 2H, J=9 Hz), 2.47 (s, 1H), 2.42-2.39 (d, 2H, J=9 Hz), 1.25 (s, 2H).

Analogues of 2-(6-amino-3-azabicyclo[3.1.0]hex-3-yl)-1,1-diphenylethanol (Compound No. 35) described below can be prepared by deprotecting appropriate amine respectively, as applicable in each case.

### a) 2-(6-Amino-3-azabicyclo[3.1.0]hex-3-yl)-1-cyclopentyl-1-phenylethanol (Compound No. 36)

¹H NMR: δ 7.42-7.16 (m, 5H), 3.01-3.00 (d, 2H), 2.79-2.75 (d, 1H), 2.44 (m, 1H), 2.0 (m, 2H), 1.88 (m, 1H), 1.58-1.06 (m, 11H).

### b) 2-[6-(Aminomethyl)-3-azabicyclo[3.1.0]hex-3-yl]-1-cyclopentyl-1-phenylethanol (Compound No. 37)

¹H NMR: δ 7.43-7.15 (m, 5H), 3.009-2.96 (m, 2H), 2.80-2.76 (d, 1H), 2.65 (ph, 1H), 2.44-2.42 (d, 2H), 2.13 (m, 2H), 1.80 (m, 1H), 1.57-1.006 (m, 12H).

### Example 14

### Biological Activity

### Radioligand Binding Assays:

The affinity of test compounds for M₂ and M₃ muscarinic receptor subtypes was determined by [³H]-N-methylscopolamine binding studies using rat heart and submandibular gland respectively as described by Moriya et al., (Life Sci., 1999, 64(25):2351-2358) with minor modifications. In competition binding studies, specific binding of [3H] NMS was also determined using membranes from Chinese hamster ovary (CHO) cells expressing cloned human M₁, M₂, M₃, M₄ and M₅ receptors. Selectivities were calculated from the Ki values obtained on these human cloned membranes.

Membrane preparation: Submandibular glands and heart were isolated and placed in ice cold homogenizing buffer (HEPES 20 mM, 10 mM EDTA, pH 7.4) immediately after sacrifice. The tissues were homogenized in 10 volumes of homogenizing buffer and the homogenate was filtered through two layers of wet gauze and filtrate was centrifuged at 500 g for 10 minutes at 4° C. The supernatant was subsequently centrifuged at 40,000 g for 20 min at 4° C. The pellet thus obtained was resuspended in assay buffer (HEPES 20 mM, EDTA 5 mM, pH 7.4) and were stored at −70° C. until the time of assay.

Ligand binding assay: The compounds were dissolved and diluted in DMSO. The membrane homogenates (150-250 μg protein) were incubated in 250 μL of assay volume (HEPES 20 mM, pH 7.4) at 24-25° C. for 3 h. Non-specific binding was determined in the presence of 1 μM atropine. The incubation was terminated by vacuum filtration over GF/B fiber filters (Wallac). The filters were then washed with ice-cold 50 mM Tris HCl buffer (pH 7.4). The filter mats were dried and bound radioactivity retained on filters was counted. The ICS₅₀ & K_{d} were estimated by using the non-linear curve fitting program using G Pad Prism software. The value of inhibition constant Ki was calculated from competitive binding studies by using Cheng & Prusoff equation (Biochem Pharmacol, 1973, 22:3099-3108),

Ki=IC₅₀/(1+L/Kd)

where L is the concentration of [³H]NMS used in the particular experiment, pki is −log [Ki].

Functional Experiments using isolated rat bladder:

### Methodology:

Animals were euthanized by overdose of thiopentone and whole bladder was isolated and removed rapidly and placed in ice cold Tyrode buffer with the following composition (mmol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder was cut into longitudinal strips (3 mm wide and 5-6 mm long) and mounted in 10 mL organ baths at 30° C., with one end connected to the base of the tissue holder and the other end connected through a force displacement transducer. Each tissue was maintained at a constant basal tension of 1 g and allowed to equilibrate for 1^{1/2} hour during which the Tyrode buffer was changed every 15-20 min. At the end of equilibration period the stabilization of the tissue contractile response was assessed with 1 μmol/L of Carbachol till a reproducible response is obtained. Subsequently a cumulative concentration response curve to carbachol (10⁻⁹ mol/L to 3×10⁻⁴ mol/L) was obtained. After several washes, once the baseline was achieved, cumulative concentration response curve was obtained in presence of NCE (NCE added 20 min prior to the second cumulative response curve.

The contractile results were expressed as % of control E max. ED50 values were calculated by fitting a non-linear regression curve (Graph Pad Prism). pKb values were calculated by the formula pKb=−log [(molar concentration of antagonist/(dose ratio−1))]

where

dose ratio=ED50 in the presence of antagonist/ED50 in the absence of antagonist.

The exemplified compounds 1-50 exhibited pKi values for M₂ from about 5 to about 8.5, from about 5 to about 7.5, and even from about 5 to about 7.1.

The exemplified compounds 1-50 exhibited pKi values for M₃ from about 6 to about 8.5, from about 6 to about 7-7, and even from about 5 to about 6.9.

1. Compounds having the structure of Formula I 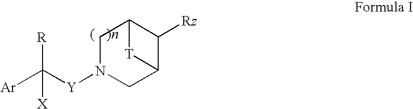
wherein
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl, cycloalkyl or aryl;
Y is —C(═O), —(C(═S), —C(═Nacyl), —C(—H(NO₂)), —C(═CH(NO₂)), —C(═C(R₁)₂) or —CH₂—;
T is —(CH₂)ₘ, —CH(Q)CH₂, —CH(Q), —CH₂—O—CH₂;
Rz is hydrogen, hydroxy, alkoxy, hydroxyalkyl, aryloxy, —CHO, —CN, alkyl, alkenyl, alkynyl, cycloalkyl, carboxy, halogen, aryl, aralkyl, acyl, heteroaryl heterocyclyl, heteroarylalkyl, heterocyclylalkyl, —(CH₂)ₖNRₓR_{y}, —SO₂R₂, —COOR₃, —C(═O)NRₓR_{y}, —NRₓR_{y}, —OC(═O)NRₓR_{y}, —NR₁C(═O)Rₓ or —NHC(═O)Rₓ; and
n is an integer from 0-2, wherein when n is zero then n represents a direct, bond);
wherein
Rₓ and R_{y} are independently selected from the group consisting of hydrogen, hydroxy, alkoxy, alkylalkenyl, alkynyl, cycloalkyl, aryl, aralkyl —SO₂R₂, carboxy, —COOR₃, heteroaryl, heterocyclyl, heteroarylalkyl or heterocyclylalkyl; or Rₓ and R_{y} may together join, to form, cycloalkyl, heteroaryl or heterocyclyl ring, wherein both Rₓ and R_{y} cannot be hydroxy at the same time);
R₁ is hydrogen, alkyl, alkenyl, alkynyl, cycloalkyl, aryl, heterocyclyl, heteroaryl, aralkyl, heteroarylalkyl or heterocyclylalkyl;
m is an integer from 0-3, wherein T represents a direct bond when m is zero;
Q is alkyl, alkenyl, alkynyl, cycloalkyl, aryl, heteroaryl, heterocyclyl, aralkyl, heteroarylalkyl of heterocyclylalkyl;
R₂ is alkyl, alkenyl, alkynyl, cycloalkyl, —NR_{g}Rₕ, aryl, aralkyl, heteroaryl, heterocyclyl, heterocyclyalkyl or heteroarylalkyl, wherein
R_{g} and Rₕ are independently selected from the group consisting of hydrogen, alkyl, alkenyl, alkynyl, cycloalkyl, aryl, aralkyl, heterocyclyl, heteroaryl, heterocyclylalkyl or heteroarylalkyl; or R_{g} and Rₕ join together to form a heterocyclyl ring;
R₃ is alkyl, alkenyl, alkynyl, cycloalkyl, aryl, aralkyl, heteroarylalkyl or heterocyclylalkyl; and
k is an integer from 1-4,
and pharmaceutically acceptable salts, pharmaceutically acceptable solvates, stereoisomers or polymorphs thereof.

2. A compound selected from;
2-[6-(Benzylamino)-3-azabicyclo[3.1.0]hex-3-yl]-1-cyclopentyl-2-oxo-1-phenylethanol (Compound No. 1),
2-[6-(Benzylamino)-3-azabicyclo[3.1.0]hex-3-yl]-1-cyclohexyl-2-oxo-1-phenylethanol (Compound No. 2),
2-(6-Amino-3-azabicyclo[3.1.0]hex-3-yl)-1-cyclopentyl-1-(4-methylphenyl)-2-oxoethanol (Compound No. 3),
Tartarate salt of 2-[6-(allylamino)-3-azabicyclo[3.1.0]hex-3-yl]-1-cyclohexyl-2-oxo-1-phenylethanol (Compound No. 4),
Tartarate salt of 2-[6-(allylamino)-3-azabicyclo[3.1.0]hex-3-yl]-1-cyclohexyl-1-(4-methylphenyl)-2-oxo ethanol (Compound No. 5),
2-[6-(Allylamino)-3-azabicyclo[3.1.0]hex-3-yl]-1-cyclopentyl-1-(4-methylphenyl)-2-oxo ethanol (Compound No. 6),
2-[6-(Allylamino)-3-azabicyclo[3.1.0]hex-3-yl]-1-cyclopentyl-2-oxo-1-phenylethanol (Compound No. 7),
Tartarate salt of 2-[6-(allylamino)-3-azabicyclo[3.1.0]hex-3-yl]-1-cyclopentyl-1-(4-fluorophenyl)-2-oxoethanol (Compound No. 8),
Tartarate salt of I-[6-(allylamino)-3-azabicyclo[3.1.0]hex-3-yl]-3-methyl-1-oxo-2-phenylbutan-2-ol (Compound No. 9),
1-Cyclopentyl-2-[6-(dimethylamino)-3-azabicyclo[3.1.0]hex-3-yl]-2-oxo-1-phenyl ethanol (Compound No. 10),
1-Cyclobutyl-2-oxo-1-phenyl-2-[6-(propylamino)-3-azabicyclo[3.1.0]hex-3-yl]ethanol (Compound No. 11),
1-Cyclohexyl-2-[6-(methylamino)-3-azabicyclo[3.1.0]hex-3-yl]-1-(4-methylphenyl)-2-oxoethanol (Compound No. 12),
1-Cyclohexyl-1-(4-fluorophenyl)-2-[6-(methylamino)-3-azabicyclo[3.1.0]hex-3-yl]-2-oxoethanol (Compound No. 13),
2-(4-Fluorophenyl)-3-methyl-1-[6-(methylamino)-3-azabicyclo[3.1.0]hex-3-yl]-1-oxo butan-2-ol (Compound No. 14),
1-Cyclopentyl-1-(4-methoxyphenyl)-2-[6-(methylamino)-3-azabicyclo[3.1.0]hex-3-yl]-2-oxoethanol (Compound No. 15),
1-Cyclohexyl-2-[6-(dimethylamino)-3-azabicyclo[3.1.0]hex-3-yl]-2-oxo-1-phenyl ethanol (Compound No. 16),
1-Cyclohexyl-2-{6-[(methylamino)methyl]-3-azabicyclo[3.1.0]hex-3-yl}-1-(4-methyl phenyl)-2-oxoethanol (Compound No. 17),
1-Cyclopentyl-2-[6-(dimethylamino)-3-azabicyclo[3.1.0]hex-3-yl]-2-oxo-1-(2-thienyl) ethanol (Compound No. 18),
1-Cyclohexyl-1-(4-fluorophenyl)-2-{6-[(methylamino)methyl]-3-azabicyclo[3.1.0] hex-3-yl}-2-oxoethanol (Compound No. 19),
2-(4-Fluorophenyl)-3-methyl-1-{6-[(methylamino)methyl]-3-azabicyclo[3.1.0]hex-3-yl}-1-oxobutan-2-ol (Compound No. 20),
2-(4-Fluorophenyl)-1-[6-(dimethylamino)-3-azabicyclo[3.1.0]hex-3-yl]-3-methyl-1-oxobutan-2-ol (Compound No. 21),
(1R)-1-Cyclopentyl-2-{6-[(methylamino)methyl]-3-azabicyclo[3.1.0]hex-3-yl}-2-oxo-1-phenylethanol (Compound No. 22),
Tartarate salt of (1R)-1-Cyclopentyl-2-[6-(methylamino)-3-azabicyclo[3.1.0]hex-3-yl]-2-oxo-1-phenylethanol (Compound No. 23),
2-[6-(Aminomethyl)-3-azabicyclo[3.1.0]hex-3-yl]-1-cyclopentyl-2-oxo-1-phenyl ethanol (Compound No. 24).
Tert-butyl ({3[cyclopentyl(hydroxy)phenylacetyl]-3-azabicyclo[3.1.0]hex-6-yl}methyl)carbamate (Compound No. 25),
2-(6-Amino-3-azabicyclo[3.1.0]hex-3-yl)-1-cyclopentyl-2-oxo-1-phenylethanol (Compound No. 26),
Tert-butyl {3-[cyclopentyl(hydroxy)phenylacetyl]-3-azabicyclo[3.1.0]hex-6-yl} carbamate (Compound No. 27),
2-(6-Amino-3-azabicyclo[3.1.0]hex-3-yl)-2-oxo 1,1-diphenylethanol (Compound No. 28),
1-Cyclohexyl-2-[6-(hydroxymethyl)-3-azabicyclo[3.1.0]hex-3-yl]-2-oxo-1-phenyl ethanol (Compound No. 29),
1-Cyclohexyl-2-{6-[ethylamino)methyl]-3-azabicyclo[3.1.0]hex-3-yl}-2-oxo-1-phenyl ethanol (Compound No. 30),
Tert-butyl{3-[cyclopentyl(hydroxy)phenylacetyl]-3-azabicyclo[3.1.0]hex-6-yl}methyl carbamate (Compound No. 31),
1-Cyclopentyl-2-[6-(methylamino)-3-azabicyclo[3.1.0]hex-3-yl]-2-oxo-1-phenyl ethanol (Compound No. 32),
Tert-butyl {3-[hydroxy(diphenyl)acetyl]-3-azabicyclo[3.1.0]hex-6-yl}methylcarbamate (Compound No. 33),
Tert-butyl [3-(2-hydroxy-2,2-diphenylethyl)-3-azabicyclo[3.1.0]hex-6-yl]carbamate (Compound No. 34),
2-(6-Amino-3-azabicyclo[3.1.0]hex-3-yl)-1,1-diphenylethanol (Compound No. 35),
2-(6-Amino-3-azabicyclo[3.1.0]hex-3-yl)-1,1-cyclopentyl-1-phenylethanol (Compound No. 36),
2-[6-(Aminomethyl)-3-azabicyclo[3.1.0]hex-3-yl]-1-cyclopentyl-1-phenylethanol (Compound No. 37),
2-{6-[(Methylamino)methyl]-3-azabicyclo[3.1.0]hex-3-yl}-2-oxo-1,1-diphenylethanol (Compound No. 38),
1-Cyclopentyl-2-{6-[(methylamino)methyl]-3-azabicyclo[3.1.0]hex-3-yl}-2-oxo-1-phenylethanol (Compound No. 39),
2-(6-Amino-3-azabicyclo[3.1.0]hex-3-yl)-1-cyclopentyl-2-oxo-1-(2-thienyl)ethanol (Compound No. 40),
2-(6-Amino-3-azabicyclo[3.1.0]hex-3-yl)-2-oxo-1-phenyl-1-(2-thienyl)ethanol (Compound No. 41),
1-Cyclohexyl-2-[6-(methylamino)-3-azabicyclo[3.1.0.]hex-3-yl]-2-oxo-1-phenyl ethanol (Compound No. 42),
1-Cyclohexyl-2-{6-[(methylamino)methyl]-3-azabicyclo[3.1.0]hex-3-yl}-2-oxo-1-phenylethanol (Compound No. 43),
1-Cyclopentyl-2-[6-(isopropylamino)-3-azabicyclo[3.1.0]hex-3-yl]2-oxo-1-phenyl ethanol (Compound No. 44),
1-Cyclohexyl-2-[6-(isopropylamino)-3-azabicyclo[3.1.0]hex-3-yl]-2-oxo-1-phenyl ethanol (Compound No. 45),
1,1,1-Trifluoro-3-[6-(methylamino)-3-azabicyclo[3.1.0]hex-3-yl]-3-oxo-2-phenyl propan-2-ol (Compound No. 46),
1,1,1-Trifluoro-3-{6-[(methylamino)methyl]-3-azabicyclo[3.1.0]hex-3-yl}-3-oxo-2-phenylpropan-2-ol (Compound No. 47),
1,1,1-Trifluoro-3-[6-(methylamino)-3-azabicyclo[3.1.0]hex-3-yl]-2-(4-methylphenyl-3-oxopropan-2-ol (Compound No. 48),
1,1,1-Trifluoro-3-{6-[(methylamino)methyl]-3-azabicyclo[3.1.0]hex-3-yl}-2-(4-methyl phenyl)-3-oxopropan-2-ol (Compound No. 49),
(1R or 1S)-2-(6-Amino-3-azabicyclo[3.1.0]hex-3-yl)-1-cyclopentyl-2-oxo-1-(2-thienyl)ethanol (Compound No. 50),
or a pharmaceutically acceptable salt, pharmaceutically acceptable solvate, stereoisomer or polymorph thereof.

3. A pharmaceutical composition comprising one or more pharmaceutically acceptable carriers, excipients or diluents and a therapeutically effective amount of one or more compounds of Formula I, 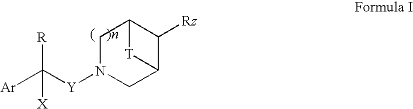
wherein
Ar is aryl, cycloalkyl, aralkyl, heteroaryl, heterocyclyl, heterocyclyalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl, cycloalkyl or aryl;
Y is —C(═O), —C(═S), —C(═Nacyl), —C(═N(NO₂)), —C(═CH(NO₂)), —C(═C(R₁)₂) or
T is —(CH₂)ₘ, —CH(Q)CH₂, —CH(Q), —CH₂—O—CH₂;
Rz is hydrogen, hydroxy, alkoxy, hydroxyalkyl, aryloxy, —CHO, —CN, alkyl, alkenyl, alkynyl, cycloalkyl, carboxy, halogen, and, aralkyl, acyl, heteroaryl, heterocyclyl, heteroarylalkyl, heterocyclylalkyl, —(CH₂)ₖNRₓR_{y}, —SO₂R₂, —COOR₃, —C(═O)NRₓR_{y}, —NRₓR_{y}, —OC(═O)NRₓR_{y}, —NR₁C(═O)Rₓ or —NHC(═O)Rₓ; and
n is an integer from 0-2, wherein when n is zero then n represents a direct bond);
wherein
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form, cycloalkyl, heteroaryl or heterocyclyl ring, wherein both Rₓ and R_{y} cannot be hydroxy at the same time);
R₁ is hydrogen, alkyl, alkenyl alkynyl, cycloalkyl, aryl, heterocyclyl, heteroaryl, aralkyl, heteroarylalkyl or heterocyclylalkyl;
m is an integer from 0-3, wherein T represents a direct bond when m is zero;
Q is alkyl, alkenyl, alkynyl, cycloalkyl, aryl, heteroaryl, heterocyclyl, aralkyl, heteroarylalkyl or heterocyclylalkyl;
R₂ is alkyl, alkenyl, alkynyl, cycloalkyl, —NR_{g}Rₕ, aryl, aralkyl, heteroaryl, heterocyclyl, heterocyclylalkyl or heteroarylalkyl, wherein
R_{g} and Rₕ are Independently selected from the group consisting of hydrogen, alkyl, alkenyl, alkynyl, cycloalkyl, aryl, aralkyl, heterocyclyl, heteroaryl, heterocyclylalkyl or heteroarylalkyl or R_{g} and Rₕ join together to form a heterocyclyl ring;
R₃ is alkyl, alkenyl, alkynyl, cycloalkyl, aryl, aralkyl, heteroarylalkyl or heterocyclylalkyl; and
k is an integer from 1-4,
or pharmaceutically acceptable salts, pharmaceutically acceptable solvates, stereoisomers or polymorphs thereof.

4. A pharmaceutical composition comprising one or more pharmaceutically acceptable carriers, excipients or diluents and one or more compound's selected from:
2-[6-(Benzylamino)-3-azabicyclo[3.1.0]hex-3-yl]-1-cyclopentyl-2-oxo-1-phenylethanol (Compound No. 1),
2-[6-(Benzylamino)-3-azabicyclo[3.1.0]hex-3-yl]-1-cyclohexyl-2-oxo-1-phenylethanol (Compound No. 2),
2-(6-Amino-3-azabicyclo[3.1.0]hex-3-yl)-1-cyclopentyl-1-(4-methylphenyl)-2-oxoethanol (Compound No. 3),
Tartarate salt of 2-[6-(allylamino)-3-azabicyclo[3.1.0]hex-3-yl]-1-cyclohexyl-2-oxo-1-phenylethanol (Compound No. 4),
Tartarate salt of 2-[6-(allylamino)-3-azabicyclo[3.1.0]hex-3-yl]-1-cyclohexyl-1-(4-methylphenyl)-2-oxo ethanol (Compound No. 5),
2-[6-(Allylamino)-3-azabicyclo[3.1.0]hex-3-yl]-1-cyclopentyl-1-(4-methylphenyl)-2-oxo ethanol (Compound No. 6),
2-[6-(Allylamino)-3-azabicyclo[3.1.0]hex-3-yl]-1-cyclopentyl-2-oxo-1-phenylethanol (Compound No. 7),
Tartarate salt of 2-[6-(allylamino)-3-azabicyclo[3.1.0]hex-3-yl]-1-cyclopentyl-1-(4-fluorophenyl)-2-oxoethanol (Compound No. 8),
Tartarate salt of 1-[6-(allylamino)-3-azabicyclo[3.1.0]hex-3-yl]-3-methyl-1-oxo-2-phenylbutan-2-ol (Compound No. 9),
1-Cyclopentyl-2-[6-(dimethylamino)-3-azabicyclo[3.1.0]hex-3-yl]-2-oxo-1-phenyl ethanol (Compound No. 10),
1-Cyclobutyl-4-oxo-1-phenyl-2-[6-(propylamino)-3-azabicyclo[3.1.0]hex-3-yl]ethanol (Compound No: 11),
1-Cyclohexyl-2-[6-(methylamino)-3-azabicyclo[3.1.0]hex-3-yl]-1-(4-methylphenyl)-2-oxoethanol (Compound No. 12),
1-Cyclohexyl-1-(4-fluorophenyl)-2-[6-(methylamino)-3-azabicyclo[3.1.0]hex-3-yl]-2-oxoethanol (Compound No. 13),
2-(4-Fluorophenyl)-3-methyl-1-[6-(methylamino)-3-azabicyclo[3.1.0]hex-3-yl]-1-oxo butan-2-ol (Compound No. 14),
1-Cyclopentyl-1-(4-methoxyphenyl)-2-[6-(methylamino)-3-azabicyclo[3.1.0]hex-3-yl]-2-oxoethanol (Compound No. 15),
1-Cyclohexyl-2-[6-(dimethylamino)-3-azabicyclo[3.1.0]hex-3-yl]-2-oxo-1-phenyl ethanol (Compound No. 16),
1-Cyclohexyl-2-{6-[(methylamino)methyl]-3-azabicyclo[3.1.0]hex-3-yl}-1-(4-methyl phenyl)-2-oxoethanol (Compound No. 17),
1-Cyclopentyl-2-[6-(dimethylamino)-3-azabicyclo[3.1.0]hex-3-yl]-2-oxo-1-(2-thienyl) ethanol (Compound No. 18),
1-Cyclohexyl-1-(4-fluorophenyl)-2-{6-[(methylamino)methyl]-3-azabicyclo[3.1.0] hex-3-yl}-2-oxoethanol (Compound No. 19),
2-(4-fluorophenyl)-3-methyl-1-{6-[(methylamino)methyl]-3-azabicyclo[3.1.0]hex-3-yl}-1-oxobutan-2-ol (Compound No. 20),
2-(4-Fluorophenyl)-1-[6-(dimethylamino)-3-azabicyclo[3.1.0]hex-3-yl]-3-methyl-1-oxobutan-2-ol (Compound No. 21),
(1R)-1-Cyclopentyl-2-{6-[(methylamino)methyl]-3-azabicyclo[3.1.0]hex-3-yl}-2-oxo-1-phenylethanol (Compound No. 22),
Tartarate salt of (1R)-1-Cyclopentyl-2-[6-(methylamino)-3-azabicyclo[3.1.0]hex-3-yl]-2-oxo-1-phenylethanol (Compound No. 23),
2-[6-(Aminomethyl)-3-azabicyclo[3.1.0]hex-3-yl]-1-cyclopentyl-2-oxo-1-phenyl ethanol (Compound No. 24),
Tert-butyl ({3-[cyclopentyl(hydroxy)phenylacetyl]-3-azabicyclo[3.1.0]hex-6-yl}methyl)carbamate (Compound No. 25),
2-(6-Amino-3-azabicyclo[3.1.0]hex-3-yl)-1-cyclopentyl-2-oxo-1-phenylethanol (Compound No. 26),
Tert-butyl {3-[cyclopentyl(hydroxy)phenylacetyl]-3-azabicyclo[3.1.0]hex-6-yl} carbamate (Compound No. 27),
2-(6-Amino-3-azabicyclo[3.1.0]hex-3-yl)-2-oxo-1,1-diphenylethanol (Compound No. 28),
1-Cyclohexyl-2-[6-(hydroxymethyl)-3-azabicyclo[3.1.0]hex-3-yl]-2-oxo-1-phenyl ethanol (Compound No. 29),
1-Cyclohexyl-2-{6-[(ethylamino)methyl]-3-azabicyclo[3.1.0]hex-3-yl}-2-oxo-1-phenyl ethanol (Compound No. 30),
Tert-butyl{3-[cyclopentyl(hydroxy)phenylacetyl]-3-azabicyclo[3.1.0]hex-6-yl}methyl carbamate (Compound No. 31),
1-Cyclopentyl-2-[6-(methylamino)-3-azabicyclo[3.1.0]hex-3-yl]-2-oxo-1-phenyl ethanol (Compound No. 32),
Tert-butyl {3-[hydroxy(diphenyl)acetyl]-3-azabicyclo[3.1.0]hex-6-yl}methylcarbamate (Compound No. 33)
Tert-butyl [3-(2-hydroxy-2,2-diphenylethyl)-3-azabicyclo[3.1.0]hex-6-yl]carbamate (Compound No. 34),
2-(6-Amino-3-azabicyclo[3.1.0]hex-3-yl)-1,1-diphenylethanol (Compound No. 35),
2-(6-Amino-3-azabicyclo[3.1.0]hex-3-yl)-1-cyclopentyl-1-phenylethanol (Compound No. 36),
2-[6-(Aminomethyl)-3-azabicyclo[3.1.0]hex-3-yl]-1-cyclopentyl-1-phenylethanol (Compound No. 37),
2-{6-[(Methylamino)methyl]-3-azabicyclo[3.1.0]hex-3-yl}-2-oxo-1,1-diphenylethanol (Compound No. 38),
1-Cyclopentyl-2-{6-[(methylamino)methyl]-3-azabicyclo[3.1.0]hex-3-yl}-2-oxo-1-phenylethanol (Compound No. 39),
2-(6-Amino-3-azabicyclo[3.1.0]hex-3-yl)-1-cyclopentyl-2-oxo-1-(2-thienyl)ethanol (Compound No. 40),
2-(6-Amino-3-azabicyclo[3.1.0]hex-3-yl)-2-oxo-1-phenyl-1-(2-thienyl)ethanol (Compound No. 41),
1-Cyclohexyl-2-[6-(methylamino)-3-azabicyclo[3.1.0]hex-3-yl]-2-oxo-1-phenyl ethanol (Compound No. 42),
1-Cyclohexyl-2-{6-[(methylamino)methyl]-3-azabicyclo[3.1.0]hex-3-yl}2-oxo-1-phenylethanol (Compound No. 43),
1-Cyclopentyl-2-[6-(isopropylamino)-3-azabicyclo[3.1.0]hex-3-yl]-2-oxo-1-phenyl ethanol (Compound No. 44),
1-Cyclohexyl-2-[6-(isopropylamino)-3-azabicyclo[3.1.0]hex-3-yl]-2-oxo-1-phenyl ethanol (Compound No. 45),
1,1,1-Trifluoro-3-[6-(methylamino)-3-azabicyclo[3.1.0]hex-3-yl]-2-oxo-1-phenyl propan-2-ol (Compound No. 46),
1,1,1-Trifluoro-3-{6-[(methylamino)methyl]-3-azabicyclo[3.1.0]hex-3-yl}-3-oxo-2-phenylpropan-2-ol (Compound No. 47),
1,1,1-Trifluoro-3-[6-(methylamino)-3-azabicyclo[3.1.0]hex-3-yl]-2-(4-methylphenyl)-3-oxopropan-2-ol (Compound No. 48),
1,1,1-Trifluoro-3-{6-[(methylamino)methyl]-3-azabicyclo[3.1.0]hex-3-yl}-2-(4-methyl phenyl)-3-oxopropan-2-ol (Compound No. 49),
(1R or 1S)-2-(6-Amino-3-azabicyclo[3.1.0]hex-3-yl)-1-cyclopentyl-2-oxo-1-(2-thienyl)ethanol (Compound No. 50),
or pharmaceutically acceptable salts, pharmaceutically acceptable solvates, stereoisomers or polymorphs, thereof.

5. A method for treatment or prophylaxis of a disease or disorder of the respiratory, urinary or gastrointestinal systems, wherein the disease or disorder is mediated through muscarinic receptors, comprising administering to an animal or human in need thereof a therapeutically effective amount of one or more compounds of Formula I, 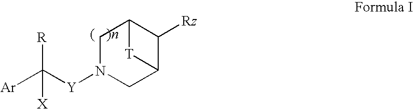
wherein
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl, cycloalkyl or aryl;
Y is —C(═O), —C(═S), C(═Nacyl), —C(—N(NO₂)), —C(═CH(NO₂)), —C(═C(R₁)₂) or —CH₂—;
T is —(CH₂)ₘ, —CH(Q)CH₂, —CH(Q), —CH₂—O—CH₂;
Rz is hydrogen, hydroxy, alkoxy, hydroxyalkyl, aryloxy, —CHO, —CN, alkyl, alkenyl, alkynyl, cycloalkyl, carboxy, halogen, aryl, aralkyl, acyl, heteroaryl, heterocyclyl, heteroaryalkyl, heterocyclyalkyl, —(CH₂)ₖNRₓR_{y}, —SO₂R₂, —COOR₃, —C(═O)NRₓR_{y}, —NRₓR_{y}, —OC(═O)NRₓR_{y}, —NR₁C(═O)Rₓ or —NHC(═O)Rₓ; and
n is an integer from 0-2, wherein when n is zero then n represents a direct bond);
wherein
Rₓ and R_{y} are: independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein, both Rₓ and R_{y} cannot be hydroxy at the same time);
R₁ is hydrogen, alkyl, alkenyl, alkynyl, cycloalkyl, aryl, heterocyclyl, heteroaryl, aralkyl, heteroarylalkyl or heterocyclylalkyl:
m is an integer from 0-3, wherein T represents a direct bond when m is zero;
Q is alkyl, alkenyl, alkynyl, cycloalkyl, aryl, heteroaryl, heterocyclyl, aralkyl heteroarylalkyl or heterocyclylalkyl;
R₂ is alkyl, alkenyl, alkynyl, cycloalkyl, —NR_{g}Rₕ, aryl, aralkyl, heteroaryl, heterocyclyl, heterocyclylalkyl or heteroarylalkyl, wherein
R_{g} and Rₕ are independently selected from the group consisting of hydrogen, alkyl, alkenyl, alkynyl, cycloalkyl, aryl, aralkyl, heterocyclyl, heteroaryl, heterocyclylalkyl or heteroarylalkyl; or R_{g} and Rₕ join together to form a heterocyclyl ring;
R₃ is alkyl, alkenyl, alkynyl, cycloalkyl, aryl, aralkyl, heteroarylalkyl or heterocyclylalkyl; and
k is an integer from 1-4,
or pharmaceutically acceptable salts, pharmaceutically acceptable, solvates, stereoisomers or polymorphs thereof.

6. The method, according to claim 5, wherein the disease or disorder is urinary incontinence, louver urinary tract symptoms (LUTS), bronchial asthma, chronic obstructive pulmonary disorders (COPD), pulmonary fibrosis, Irritable bowel, syndrome, obesity, diabetes or gastrointestinal hyperkinesis.

7. A process of preparing a compound of Formula IV, V, VI or VII, or a pharmaceutically acceptable salt, pharmaceutically acceptable solvate, stereoisomer or polymorph thereof, comprising the steps of:
a) condensing, a compound of Formula II 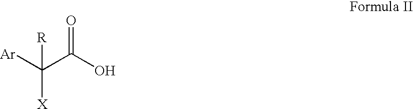
with a compound of Formula III (wherein p is 0 or 1, Rₖ is R_{y} or P) 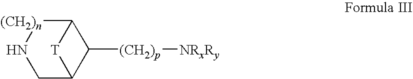
to form a compound of Formula IV, 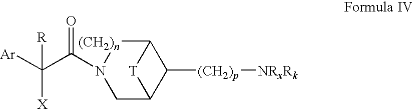
and
b) i) (Path a) optionally deprotecting the compound of Formula IV (wherein Rₖ is P) to form a compound of formula V, 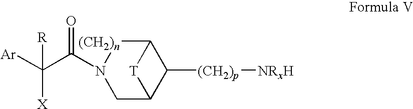
or
ii) (Path b)
(A) optionally reducing the compound of Formula IV (wherein Rₓ is —(CH₂)_{q}CH═CH₂, wherein q is an integer from 1 to 3) to form a compound of Formula VI, 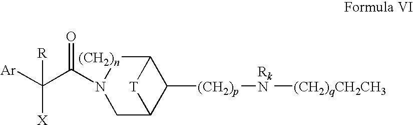
and
(B) optionally deprotecting the compound of Formula VI (wherein Rₖ is P) to form the compound of Formula VII, 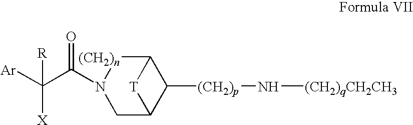
wherein,
Ar is aryl, cycloalkyl, aralkyl, heteroaryl, heterocyclyl, heterocyclylalkyl or heteroarylalkyl;
X is alkylalkenyl, alkynyl, cycloalkyl, aryl, heteroaryl, heterocyclyl aralkyl, heterocyclylalkyl or heteroarylalkyl;
R is hydrogen, hydroxy, alkoxy, aryloxy, hydroxyalkyl, —NRₓR_{y}, halogen, alkyl, alkenyl, alkynyl cycloalkyl or aryl;
T is —(CH₂)ₘ, —CH(Q)CH₂, —CH(Q), —CH₂—O—CH₂;
P is —C(═O)OC(CH₃)₃, —C(═O)C(CH₃)₂CHBr₂ or —C(═O)C(CH₃)₂CCl₃;
n is an integer from 0-2, wherein when n is zero then n represents a direct bond); and
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time.

8. The process of claim 7, wherein the compound of Formula II is condensed with a compound of Formula III to form, a compound of Formula IV with one or more condensing agents, and one or more bases.

9. (canceled)

10. (canceled)

11. (canceled)

12. The process of claim 7, wherein the compound of formula IV is deprotected to form a compound of Formula V in the presence of one or more: acids or one or more supernucleophiles.

13. (canceled)

14. (canceled)

15. The process of claim 7, wherein the compound of Formula IV can be reduced to form compounds of Formula VI with one or more reducing agents.

16. (canceled)

17. The process of claim 7, wherein the compound of Formula VI is deprotected to form the compound of Formula VII in the presence of one or more acids or one or more supernucleophiles.

18. (canceled)

19. (canceled)

20. The process of claim 17, wherein
a) Rₖ is P and P is —C(═O)OC(CH₃)₃ and the one or more acids are selected from hydrochloric acid, trifluoroacetic acid or mixtures thereof;
b) Rₖ is P and P is —C(═O)OC(CH₃)₂CHBr₂ and the one or more acids are selected from hydrobromic acid, hydrochloric acid or mixtures thereof; or
c) Rₖ is P and P is —C(═O)OC(CH₃)₂ CHCl₂; and the one or more supernucleophiles are selected from lithium cobalt (I) phthalocyanine, zinc and acetic acid, cobalt phthalocyanine or mixtures thereof.

21. A process of preparing a compound of Formula IX, X or XII, or a pharmaceutically acceptable salt, pharmaceutically acceptable solvate, stereoisomer or polymorph thereof, comprising the steps of:
a) condensing a compound of Formula II 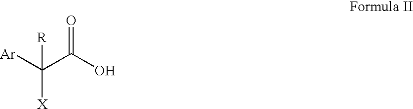
with a compound of Formula VIII 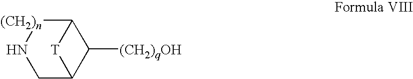
to form a compound of Formula IX; 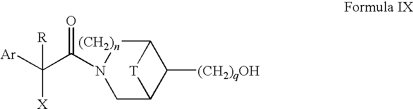
b) optionally O-derivatizing the compound of Formula IX to form a compound of Formula X (wherein P₁ is mesyl or tosyl), 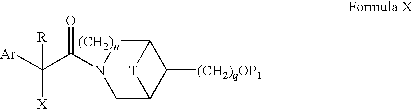
and
c) optionally reacting the compound of Formula X with a compound of Formula XI
HNRₓR_{y} Formula XI
to form a compound of Formula XII, 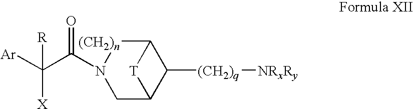
wherein,
Ar is aryl, cycloalkyl, aralkyl, heteroaryl, heterocyclyl, heterocyclylalkyl or heteroarylalkyl;
X is alkyl, alkenyl, alkynyl, cycloalkyl, aryl, heteroaryl, heterocyclyl, aralkyl, heterocyclylalkyl or heteroarylalkyl;
T is —(CH₂)ₘ, —CH(Q)CH₂, —CH(Q), —CH₂—O—CH₂;
n is an integer from 0-2, wherein n represents a direct bond when n is zero;
q is an integer from 1 to 3; and
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time.

22. The process of claim 21, wherein the compound of Formula II is condensed with the compound of Formula VIII with one or more condensing agents.

23. (canceled)

24. The process of claim 21, wherein the compound of formula II is condensed with a compound of Formula III in the presence of one or more bases.

25. (canceled)

26. The process of claim 21, wherein the compound of Formula IX is O-derivatized in the presence of one or more bases.

27. (canceled)

28. A process of preparing a compound of Formula XIV or Formula XV, or a pharmaceutically acceptable salt, pharmaceutically acceptable solvate, stereoisomer or polymorph thereof comprising the steps of;
a) reacting a compound of Formula XIII 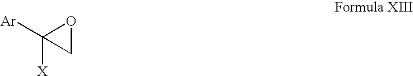
with a compound of Formula III 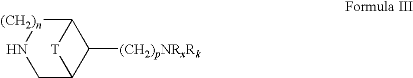
to form a compound of Formula XIV, 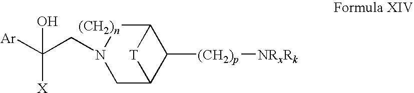
and
b) optionally deprotecting the compound of Formula XIV (wherein Rₖ is P) to form a compound of Formula XV, 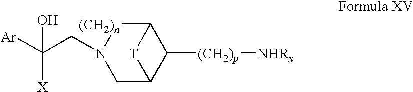
wherein
Ar is aryl, cycloalkyl, aralkyl, heteroaryl, heterocyclyl, heterocyclylalkyl or heteroarylalkyl;
X is alkyl, alkenyl alkynyl, cycloalkyl, aryl, heteroaryl, heterocyclyl, aralkyl, heterocyclylalkyl or heteroarylalkyl;
T is —(CH₂)ₘ, —CH(Q)CH₂, —CH(Q), —CH₂—O—CH₂;
n is an integer train 0-2, wherein n represents a direct bond when n is zero;
Rₓ and R_{y} are independently selected from the group consisting of hydrogen, hydroxy, alkoxy, alkyl, alkenyl, alkynyl, cycloalkyl, aryl, aralkyl, —SO₂R₂, carboxy, —COOR₃, heteroaryl, heterocyclyl, heteroarylalkyl or heterocyclylalkyl; or Rₓ and R_{y} may together join to form cycloalkyl, heteroaryl or heterocyclyl ring, wherein both Rₓ and R_{y} cannot be hydroxy at the same time;
p is 0 or 1; and
P is —C(═O)OC(CH₃)₃, —C(═O)C(CH₃)₂CHBr₂ or —C(═O)C(CH₃)₂CCl₃).

29. The process of claim 28, wherein the compound of Formula XIII is reacted with the compound of Formula III In the presence of one or more bases.

30. (canceled)

31. The process of claim 29, wherein the compound of Formula XIV is deprotected in the presence of one or more acids or one or more supernucleophiles.

32. (canceled)

33. (canceled)

34. The process of claim 31, wherein
a) Rₖ is P and P is —C(═O)OC(CH₃)₃ and the one or more acids are selected from hydrochloric acid, trifluoroacetic acid or mixtures thereof;
Rₖ is P and P is —C(═O)OC(CH₃)₂CHBr₂ and the one or more acids are selected from hydrobromic acid, hydrochloric acid or mixtures thereof; or
c) Rₖ is P and P is —C(═O)OC(CH₃)₂CHCl₂ and die one or more supernucleophiles are selected from lithium cobalt (I) phthalocyanine, zinc and acetic acid, cobalt phthalocyanine or mixtures thereof.
